(19) United States
(12) Patent Application Publication
(10) Pub. No.: US 2026/0263635 A1
(43) Pub. Date: Sep. 10, 2026

(54) BASE EDITING APPROACHES FOR CORRECTING THE IVS2-1...

(71) Applicants: INSTITUT NATIONAL DE LA SANTÉ ET DE LA RECHERCHE MÉDICALE, PARIS (FR); UNIVERSITÉ PARIS CITÉ, PARIS (FR); ASSISTANCE PUBLIQUE-HÔPITAUX DE PARIS (APHP), PARIS (FR); FONDATION IMAGINE, PARIS (FR)

(72) Inventors: Annarita MICCIO, PARIS (FR); Giulia HARDOUIN, PARIS (FR)

(21) Appl. No.: 18/881,516

(22) PCT Filed: Jul. 21, 2023
(86) PCT No.: PCT/EP2023/070283

§ 371 Date: Jan. 6, 2025

(30) Foreign Application Priority Data
Jul. 22, 2022 (EP) 22306104.5

(51) Int. Cl. A61K 48/00; A61K 38/42; A61P 7/06; C12N 9/22; C12N 9/78; C12N 15/11

(52) U.S. Cl. CPC A61K 48/005; A61K 38/42; A61P 7/06; C12N 9/226; C12N 9/78; C12N 15/111; C07K 2319/80; C12N 2310/20; C12Y 305/04004

(57) ABSTRACT

IVS2-1 (G>A) is one of the most common β⁰-thalassemic mutations in the Middle East³. This point mutation occurs at the first nucleotide of the second intron of HBB and disrupts the splice donor site. It results in the production of two abnormally spliced mRNAs⁴. The first mRNA is characterized by a partial retention of the second intron, whereas the second mRNA consists of the first exon spliced directly with the third one. Importantly, none of them lead to the production of β-globin. Here, the inventors exploited adenine base-editors (ABEs) to correct the IVS2-1 (G>A) mutation in HSPCs from β-thalassemia patients and demonstrated the potential of this strategy to correct the pathological phenotype observed during erythroid differentiation. In particular the inventors demonstrated that reverting the IVS2-1 (G>A) mutation using base editing corrected in vitro the β-thalassemic cell phenotype in terms of erythroid differentiation, enucleation, RBC size and apoptosis. The present invention thus relates to base editing approaches for the treatment of β-thalassemia, including sickle β-thalassemia.

### FIELD OF THE INVENTION

The present invention is in the field of medicine, in particular haematology.

### BACKGROUND OF THE INVENTION

β-globin chains together with α-globin chains compose the adult hemoglobin (HbA) tetramer. β-thalassemia is a monogenic recessive disease caused by a variety of mutations affecting the synthesis of the adult hemoglobin β-chains. It is a highly prevalent hemoglobinopathy with 68,000 affected children annually worldwide¹. Patients were originally concentrated in Asia, India and the Mediterranean region but due to recent population movements, β-thalassemia is now widely spread in Europe and North America¹. Point mutations or deletions in the β-globin gene (HBB) locus reduce (β⁺) or abolish (β⁰) the production of β-globin chains. The imbalance between α- and β-globin production, leads to the precipitation of uncoupled α-globins, which causes erythroid cell death, ineffective erythropoiesis and severe anemia². Depending on their genotype, the clinical phenotype can vary from mild to severe anemia, known as β-thalassemia major (typically associated with a β⁰/β⁰ genotype). In the most severe cases, patients are transfusion-dependent and require an iron-chelation therapy to alleviate iron overload due to chronic transfusions.

IVS2-1 (G>A) is one of the most common β⁰-thalassemic mutations in the Middle East, representing around 30% of β-thalassemic mutations found in in Iran and Kuwait³. This point mutation occurs at the first nucleotide of the second intron of HBB and disrupts the splice donor site. It results in the production of two abnormally spliced mRNAs⁴. The first mRNA is characterized by a partial retention of the second intron, whereas the second mRNA consists of the first exon spliced directly with the third one. Importantly, none of them lead to the production of β-globin.

The only definitive cure for β-thalassemia patients is the transplantation of allogeneic hematopoietic stem cells (HSCs), but this treatment is limited by the availability of HLA-compatible donors. Gene therapy approaches based on the transplantation of autologous, genetically modified HSCs have been investigated as a treatment option for patients lacking a compatible donor⁵.

Genome editing technology has been exploited to develop therapeutic approaches for β-hemoglobinopathies. In the case of IVS2-1 (G>A) mutation, correction of the abnormal mRNA splicing would require the precise restoration of the splice donor site. Approaches stimulating gene correction in the presence of a WT single-stranded DNA donor (containing a wild-type β-globin gene fragment) through peptide nucleic acid (PNA) binding enabled precise correction of the mutation, but with very low frequencies (<1%) 6. Other approaches use designer nucleases, such as the CRISPR/Cas9 nuclease system that induces DNA double-strand breaks (DSBs) via a single guide RNA (gRNA) complementary to a specific genomic target. The DSB can be repaired via homologous-directed repair (HDR), by providing a donor DNA template containing the wild type sequence, allowing direct gene correction of the mutation. However, in bona fide HSCs, the target cell population in gene therapy, HDR-mediated gene correction is known to be poorly efficient⁷. Furthermore, HSCs are highly sensitive to DNA double-strand breaks (DSBs)⁸—especially in cases of multiple on-targets or concomitant on-target and off-target events. Even when highly specific gRNAs are used, Cas9-gRNA treatment of human hematopoietic/stem progenitor cells (HSPCs) induces a DNA damage response that can lead to apoptosis^{9,10}. CRISPR-Cas9 can cause p53-dependent cell toxicity and cell cycle arrest, resulting in the negative selection of cells with a functional p53 pathway¹¹. Furthermore, the generation of several on-target DSBs, simultaneous on-target and off-target DSBs, or even a single on-target DSB is associated with a risk of deletion, inversion and translocation¹²⁻¹⁵.

Base editing, a new CRISPR/Cas9-derived genome editing tool, allows precise DNA repair in bona fide HSCs¹⁶ without the occurrence of DSBs. Adenine base editors (ABE) and cytosine base editors (CBE) contain a Cas9 nickase and a deaminase, and permit the insertion of A>G and C>T mutations, respectively¹⁷. Base editing has been exploited to correct a β-thalassemia-causing mutation in the HBB promoter^{16,18}.

### SUMMARY OF THE INVENTION

The present invention is defined by the claims. In particular, the present invention relates to base editing approaches for the treatment of β-thalassemia.

### DETAILED DESCRIPTION OF THE INVENTION

Here, the inventors exploited adenine base-editors (ABEs) to correct the IVS2-1 (G>A) mutation in HSPCs from β-thalassemia patients and demonstrated the potential of this strategy to correct the pathological phenotype observed during erythroid differentiation.

### Definitions

As used herein, the term “β-thalassemia” refers to a hemoglobinopathy that results from an altered ratio of α-globin to β-like globin polypeptide chains resulting in the underproduction of normal hemoglobin tetrameric proteins and the precipitation of free, unpaired α-globin chains.

As used herein, the term “sickle β-thalassemia” refers to a particular form of β-thalassemia wherein the patient has a mutation in each copy of their HBB gene: one that causes red blood cells to form a “sickle” or crescent shape and a second that is associated with beta thalassemia, a blood disorder that reduces the production of hemoglobin. Clinical manifestations depend on the amount of residual beta globin chains production, and are similar to sickle cell disease, including anemia, vascular occlusion and its complications, acute episodes of pain, acute chest syndrome, pulmonary hypertension, sepsis, ischemic brain injury, splenic sequestration crisis and splenomegaly.

As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin-CD34+CD38−CD90+CD45RA−, Lin−CD34+CD38−CD90−CD45RA−, Lin−CD34+CD38+IL−3aloCD45RA−, and Lin−CD34+CD38+CD10+ (Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

As used herein, the term “peripheral blood cells” refer to the cellular components of blood, including red blood cells, white blood cells, and platelets, which are found within the circulating pool of blood. In some embodiments, the eukaryotic cell is a bone marrow derived stem cell.

As used herein the term “bone marrow-derived stem cells” refers to stem cells found in the bone marrow. Stem cells may reside in the bone marrow, either as an adherent stromal cell type that possess pluripotent capabilities, or as cells that express CD34 or CD45 cell-surface protein, which identifies hematopoietic stem cells able to differentiate into blood cells.

As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment.

As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1); agonists of the chemokine (C—C motif) receptor 1 (CCR1), such as chemokine (C—C motif) ligand 3 (CCL3, also known as macrophage inflammatory protein-1α (Mip-1α)); agonists of the chemokine (C—X—C motif) receptor 1 (CXCR1) and 2 (CXCR2), such as chemokine (C—X—C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells for use in transplantation, organ repair or regeneration, or treatment of disease.

As used herein, the term “isolated cell” refers to a cell that has been removed from an organism in which it was originally found, or a descendant of such a cell. Optionally the eukaryotic cell has been cultured in vitro, e.g., in the presence of other cells. Optionally the eukaryotic cell is later introduced into a second organism or reintroduced into the organism from which it (or the cell from which it is descended) was isolated. As used herein, the term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “nucleic acid molecule” or “polynucleotide” refers to a DNA molecule (for example, but not limited to, a cDNA or genomic DNA). The nucleic acid molecule can be single-stranded or double-stranded.

As used herein, the term “isolated” when referring to nucleic acid molecules or polypeptides means that the nucleic acid molecule or the polypeptide is substantially free from at least one other component with which it is associated or found together in nature.

As used herein, the term “complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, the term “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein, the term “hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

As used herein the term “wild type” is a term of the art understood by skilled persons and means the typical form of an organism, strain, gene or characteristic as it occurs in nature as distinguished from mutant or variant forms.

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue.

As used herein, the term “mutagenesis” refers to the introduction of mutations into a polynucleotide sequence. According to the present invention mutations are introduced into a target DNA molecule.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. A variant molecule can have entire sequence identity with the original parent molecule, or alternatively, can have less than 100% sequence identity with the parent molecule. For example, a variant of a sequence can be a second sequence that is at least 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100% identical in sequence compare to the original sequence.

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “alpha globin” or “α-globin” has its general meaning in the art and refers to protein that is encoded in human by the HBA1 and HBA2 genes. The human alpha globin gene cluster located on chromosome 16 spans about 30 kb and includes seven loci: 5′-zeta-pseudozeta-mu-pseudoalpha-1-alpha-2-alpha-1-theta-3′. The alpha-2 (HBA2) and alpha-1 (HBA1) coding sequences are identical. These genes differ slightly over the 5′ untranslated regions and the introns, but they differ significantly over the 3′ untranslated regions. The ENSEMBL IDs (i.e. the gene identifier number from the Ensembl Genome Browser database) for HBA1 and HBA2 are ENSG00000206172 and ENSG00000188536 respectively.

As used herein, the term “beta globin” or “β-globin” has its general meaning in the art and refers to a globin protein, which along with alpha globin (HBA), makes up the most common form of haemoglobin (Hb) in adult humans. Normal adult human Hb is a heterotetramer consisting of two alpha chains and two beta chains. HBB is encoded by the HBB gene on human chromosome 11. It is 146 amino acids long and has a molecular weight of 15,867 Da.

As used herein, the term “IVS2-1 (G>A) mutation” or “HBB:c.315+1G>A” has its general meaning in the art and refers to one of the most common β-thalassemic mutations in the Middle East³. This point mutation disrupts the splice donor site of the second intron of HBB leading to the absence of β-globin production.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Any method known in the art can be used to measure the expression of the gene (e. g. HPLC analysis of protein and RT-qPCR analysis of mRNA.) Typically, said methods are described in the EXAMPLE.

As used herein, the expression “restoring the normal expression of β-globin” indicates that the expression of β-globin is restored to at approximately the same level as for an eukaryotic cell that does not carry the IVS2-1 (G>A) mutation (i.e. an eukaryotic carrying the wild type HBB gene).

As used herein, the term “derived from” refers to a process whereby a first component (e.g., a first molecule), or information from that first component, is used to isolate, derive or make a different second component (e.g., a second molecule that is different from the first).

As used herein, the term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

As used herein, the term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the “linked” as used herein refers to the attachment of two or more entities to form one entity. A conjugate encompasses both peptide-small molecule conjugates as well as peptide-protein/peptide conjugates.

As used herein, the term “base-editor” refers to fusion protein comprising a defective CRISPR/Cas nuclease linked to a deaminase polypeptide. Two classes of base-editors—“cytosine base-editors” (CBEs) and “adenine base-editors” (ABEs)—can be used to generate single base pair edits without double stranded breaks. Typically, base-editor are created by fusing the defective CRISPR/Cas nuclease to a deaminase.

As used herein, the term “deaminase” refers to an enzyme that catalyses a deamination reaction. The term “deamination”, as used herein, refers to the removal of an amine group from one molecule. In some embodiments, the deaminase is a “cytidine deaminase”, catalysing the hydrolytic deamination of cytidine or deoxycytidine to uracil or deoxyuracil, respectively. In some embodiments, the deaminase is an “adenosine deaminase”, catalysing the hydrolytic deamination of adenosine to inosine, which is treated like guanosine by the cell, creating an A to G (or T to C) change.

As used herein, the term “nuclease” includes a protein (i.e. an enzyme) that induces a break in a nucleic acid sequence, e.g., a single or a double strand break in a double-stranded DNA sequence.

As used herein, the term “CRISPR/Cas nuclease” has its general meaning in the art and refers to segments of prokaryotic DNA containing clustered regularly interspaced short palindromic repeats (CRISPR) and associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG for S. pyogenes Cas9) protospacer adjacent motif (PAM) to specify the cut site (the 3rd or the 4th nucleotide upstream from PAM).

As used herein, the term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, the term “Cas9” refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1). Typically the Cas9 nuclease comprises the amino acid sequence as set forth in SEQ ID NO: 1.

Cas9 sequence
SEQ ID NO: 1:
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKERGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

As used herein, the term “defective CRISPR/Cas nuclease” refers to a CRISPR/Cas nuclease having lost at least one nuclease domain.

As used herein, the term “nickase” has its general meaning in the art and refers to an endonuclease which cleaves only a single strand of a DNA duplex. Accordingly, the term “Cas9 nickase” refers to a nickase derived from a Cas9 protein, typically by inactivating one nuclease domain of Cas9 protein.

As used herein, the term “guide RNA molecule” generally refers to an RNA molecule (or a group of RNA molecules collectively) that can bind to a Cas9 protein and target the Cas9 protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to using the CRISPR system as disclosed herein.

As used herein, the term “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “ribonucleoprotein complex,” or “ribonucleoprotein particle” refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid, it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” is meant a sufficient amount of population of cells to treat the disease at a reasonable benefit/risk ratio applicable to any medical treatment. It will be understood that the total usage compositions of the present invention will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of cells, and like factors well known in the medical arts. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the cells with the desired specificity, on the age and weight of the recipient, and on the severity of the targeted condition. This number of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. Typically, the minimal dose is 2 millions of cells per kg. Usually 2 to 20 millions of cells are injected in the subject. The desired purity can be achieved by introducing a sorting step. For uses provided herein, the cells are generally in a volume of a liter or less, can be 500 ml or less, even 250 ml or 100 ml or less. The clinically relevant number of cells can be apportioned into multiple infusions that cumulatively equal or exceed the desired total amount of cells.

### Methods of the Present Invention:

The present invention relates to a method of restoring the normal expression of β-globin in a eukaryotic cell carrying the IVS2-1 (G>A) mutation comprising the step of contacting the eukaryotic cell with a gene editing platform that consists of a (a) at least one adenine base-editor (ABE) and (b) least one guide RNA molecule for guiding the adenine base-editor to at least one target sequence comprising the IVS2-1 (G>A) mutation and thereby restoring the production of β-globin in the eukaryotic cell.

In some embodiments, the eukaryotic cell is selected from the group consisting of hematopoietic progenitor cells, hematopoietic stem cells (HSCs), pluripotent cells (i.e. embryonic stem cells (ES) and induced pluripotent stem cells (iPS)). Typically, the eukaryotic cell results from a stem cell mobilization.

In some embodiments, the eukaryotic cell is homozygous or heterozygous for the IVS2-1 (G>A) mutation.

In some embodiments, the adenine base-editor of the present invention comprises a defective CRISPR/Cas nuclease. The sequence recognition mechanism is the same as for the non-defective CRISPR/Cas nuclease. Typically, the defective CRISPR/Cas nuclease of the invention comprises at least one RNA binding domain. The RNA binding domain interacts with a guide RNA molecule as defined hereinafter. However, the defective CRISPR/Cas nuclease of the invention is a modified version with no nuclease activity. Accordingly, the defective CRISPR/Cas nuclease specifically recognizes the guide RNA molecule and thus guides the base-editor to its target DNA sequence.

In some embodiments, the defective CRISPR/Cas nuclease can be modified to increase nucleic acid binding affinity and/or specificity, alter an enzymatic activity, and/or change another property of the protein. In some embodiments, the nuclease domains of the protein can be modified, deleted, or inactivated. In some embodiments, the protein can be truncated to remove domains that are not essential for the function of the protein. In some embodiments, the protein is truncated or modified to optimize the activity of the RNA binding domain.

In some embodiments, the CRISPR/Cas nuclease consists of a mutant CRISPR/Cas nuclease i.e. a protein having one or more point mutations, insertions, deletions, truncations, a fusion protein, or a combination thereof. In some embodiments, the mutant has the RNA-guided DNA binding activity, but lacks one or both of its nuclease active sites. In some embodiments, the mutant comprises an amino acid sequence having at least 50% of identity with the wild type amino acid sequence of the CRISPR/Cas nuclease. Various CRISPR/Cas nucleases can be used in this invention. Non-limiting examples of suitable CRISPR/CRISPR/Cas nucleases include Cas3, Cas4, Cas5, Case (or CasD), Cas6, Cashe, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761 WO2014144592, WO2013176772, US20140273226, and US20140273233, the contents of which are incorporated herein by reference in their entireties.

In some embodiments, the CRISPR/Cas nuclease is derived from a type II CRISPR-Cas system. In some embodiments, the CRISPR/Cas nuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina, inter alia.

In some embodiments, the CRISPR/Cas nuclease is a mutant of a wild type CRISPR/Cas nuclease (such as Cas9) or a fragment thereof. In some embodiments, the CRISPR/Cas nuclease is a mutant Cas9 protein from S. pyogenes.

Methods for generating a Cas9 protein (or a fragment thereof) having an inactive DNA cleavage domain are known (See, e.g., Jinek et al., Science. 337:816-821 (2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of S. pyogenes Cas9 (Jinek et al., Science. 337:816-821 (2012); Qi et al., Cell. 28; 152(5):1173-83 (2013).

In some embodiments, the CRISPR/Cas nuclease of the present invention is nickase and more particularly a Cas9 nickase i.e. the Cas9 from S. pyogenes having one mutation selected from the group consisting of D10A and H840A. In some embodiments, the nickase of the present invention comprises the amino acid sequence as set forth in SEQ ID NO: 2 or SEQ ID NO:3.

> S. pyogenes nCas9 Protein Sequence having
the D10A mutation
SEQ ID NO: 2
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLFDDKVMKQ
LKRRRYTGWGRLSRKLINGIRDKQSGKTILDELKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKEDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
> S. pyogenes nCas9 Protein Sequence having
the H840A mutation
SEQ ID NO: 3
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In some embodiments, the Cas9 variants having mutations other than D10A or H840A are used, which e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain). In some embodiments, variants of dCas9 are provided which are at least about 70% identical, at least about 80% identical, at least about 90% identical, at least about 95% identical, at least about 98% identical, at least about 99% identical, at least about 99.5% identical, or at least about 99.9% to SEQ ID NO: 2 or 3. In some embodiments, variants of dCas9 are provided having amino acid sequences which are shorter, or longer than SEQ ID NO: 2 or 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

According to the present invention, the second component of the adenine base-editor herein disclosed comprises a non-nuclease DNA modifying enzyme that is an adenosine deaminase.

In some embodiments, the adenosine deaminase is an ADAT family deaminase. In some embodiments, the adenosine deaminase is a TadA deaminase. In some embodiments, the adenosine deaminase is a Staphylococcus aureus TadA, a Bacillus subtilis TadA, a Salmonella typhimurium TadA, a Shewanella putrefaciens TadA, a Haemophilus influenzae F3031 TadA, a Caulobacter crescentus TadA, or a Geobacter sulfurreducens TadA, or a fragment thereof. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5,6, 7, 8, 9, 10, 11, 12, 13, 14, 15, 6, 17, 18, 19, or 20 C-terminal amino acid residues relative to the full length ecTadA. In some embodiments, the TadA deaminase is TadA*7.10. In some embodiments, the TadA deaminase is a TadA*8 variant. For example, deaminase are described in International PCT Application WO2018/027078, WO2017/070632, WO/2020/168132, WO/2021/050571 each of which is incorporated herein by reference for its entirety. Also, see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); Komor, A. C., et al., “Improved base excision repair inhibition and bacteriophage Mu Gam protein yields C: G-to-T: A base editors with higher efficiency and product purity” Science Advances 3:eaao4774 (2017)), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference. An exemplary amino acid sequence for the wild type TadA(wt) adenosine deaminase is shown as SEQ ID NO:4. In some embodiments, the amino acid sequence of the adenosine deaminase comprises at least 90% sequence identity to SEQ ID NO:4. In some embodiments, the amino acid sequence of the adenosine deaminase comprises the modification at position 82 as numbered in SEQ ID NO:4. In some embodiments, the amino acid sequence comprises of the adenosine deaminase comprises a V82S modification, wherein position 82 is as numbered in SEQ ID NO:4. In some embodiments, the amino acid sequence of the adenosine deaminase comprises the modification at position 166 as numbered in SEQ ID NO:4. In some embodiments, the amino acid sequence of the adenosine deaminase comprises a T166R modification, wherein position 166 is as numbered in SEQ ID NO:4. In some embodiments, the amino acid sequence of the adenosine deaminase comprises modifications at positions 82 and 166 as numbered in SEQ ID NO:4. In some embodiments, the amino acid sequence of the adenosine deaminase comprises V82S and T166R modifications, wherein positions 82 and 166 are as numbered in SEQ ID NO:4. In some embodiments, the adenosine deaminase variant further comprises one or more of the following alterations: Y147T, Y147R, Q154S, Y123H, and Q154R. In some embodiments, the adenosine deaminase variant comprises a combination of alterations selected from the group consisting of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+ Q154R. In some embodiments, the adenosine deaminase variant is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In some embodiments, the adenosine deaminase is provided as a single (e.g., provided as a monomer) TadA variant as described above. In some embodiments, adenosine deaminase is provided as a heterodimer of a wild-type TadA (TadA(wt)) linked to a TadA variant as described above.

> TadA sequence
SEQ ID NO: 4
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD

In some embodiments, the adenosine deaminase is fused to the N-terminus of the defective CRISPR/Cas nuclease. In some embodiments, the adenosine deaminase is fused to the C-terminus of the defective CRISPR/Cas nuclease. In some embodiments, the defective CRISPR/Cas nuclease and the adenosine deaminase are fused via a linker. In some embodiments, the linker comprises a (GGGGS)n (SEQ ID NO:5), a (G)n, an (EAAAK)n (SEQ ID NO: 6), a (GGS)n, an SGSETPGTSESATPES (SEQ ID NO:7) motif (see, e.g., Guilinger J P, Thompson D B, Liu D R. Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65(10):1357-69, the entire contents of which are incorporated herein by reference.

In some embodiments, the fusion protein may comprise additional features. Other exemplary features that may be present are localization sequences, such as nuclear localization sequences (NLS), cytoplasmic localization sequences, export sequences, such as nuclear export sequences, or other localization sequences, as well as sequence tags that are useful for solubilization, purification, or detection of the fusion proteins. Suitable localization signal sequences and sequences of protein tags are provided herein, and include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable features will be apparent to those of skill in the art.

Various adenine base-editors are known in the art (see e.g. Improving cytidine and adenine base-editors by expression optimization and ancestral reconstruction. Nat Biotechnol. 2018 May 29) and typically include those described in Table A.

TABLE A
some exemplary base-editors

| Base- | |
|---|---|
| editor | References |
| ABEmax | Improving cytidine and adenine base-editors by expression optimization and |
| | ancestral reconstruction. Nat Biotechnol. 2018 May 29. pii: nbt.4172. doi: |
| | 10.1038/nbt.4172. |
| ABE- | Walton R T, Christie K A, Whittaker M N, Kleinstiver B P. Unconstrained |
| SpRY | genome targeting with near-PAMless engineered CRISPR-Cas9 variants. |
| | Science. 2020 Apr. 17; 368(6488): 290-296. doi: 10.1126/science.aba8853. |
| | Epub 2020 Mar. 26. PMID: 32217751; PMCID: PMC7297043. |
| ABE8e | Richter, M. F., Zhao, K. T., Eton, E. et al. Phage-assisted evolution of |
| | an adenine base editor with improved Cas domain compatibility and activity. |
| | Nat Biotechnol 38, 883-891 (2020). |
| NG- | Richter, M. F., Zhao, K. T., Eton, E. et al. Phage-assisted evolution of |
| ABE8e | an adenine base editor with improved Cas domain compatibility and activity. |
| | Nat Biotechnol 38, 883-891 (2020). |

In some embodiments, the adenine base-editor consists of the amino acid sequence as set forth in SEQ ID NO:8 (SpRY-ABE8e) or SEQ ID NO:9 (NG-ABE8e).

> amino acid sequence of SpRY-ABE8e
SEQ ID NO: 8
MKRTADGSEFESPKKKRKVSEVEFSHEYWMRHALTLAKRARDEREVPVGA
VLVLNNRVIGEGWNRAIGLHDPTAHAEIMALRQGGLVMQNYRLIDATLYV
TFEPCVMCAGAMIHSRIGRVVFGVRNSKRGAAGSLMNVLNYPGMNHRVEI
TEGILADECAALLCDFYRMPRQVFNAQKKAQSSINSGGSSGGSSGSETPG
TSESATPESSGGSSGGSDKKYSIGLAIGTNSVGWAVITDEYKVPSKKFKV
LGNTDRHSIKKNLIGALLFDSGETAERTRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNEDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRENASLGTYHDLLKIIKDKDELDNEENEDILEDIVLTLTLFEDREMIEE
RLKTYAHLEDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVR
KVLSMPQVNIVKKTEVQTGGFSKESIRPKRNSDKLIARKKDWDPKKYGGF
LWPTVAYSVLVVAKVEKGKSKKLKSVKELLGITIMERSSFEKNPIDFLEA
KGYKEVKKDLIIKLPKYSLFELENGRKRMLASAKQLQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTRLGAPRAFKYFDTTIDPK
QYRSTKEVLDATLIHQSITGLYETRIDLSQLGGDSGGSKRTADGSEFEPK
KKRKVGSGATNFSLLKQAGDVEENPGPMVSKGEELFTGVVPILVELDGDV
NGHKESVSGEGEGDATYGKLTLKFICTTGKLPVPWPTLVTTLTYGVQCFS
RYPDHMKQHDFFKSAMPEGYVQERTIFFKDDGNYKTRAEVKFEGDTLVNR
IELKGIDFKEDGNILGHKLEYNYNSHNVYIMADKQKNGIKVNFKIRHNIE
DGSVQLADHYQQNTPIGDGPVLLPDNHYLSTQSALSKDPNEKRDHMVLLE
FVTAAGITLGMDELYKSGGSPKKKRKV*
> Amino acid sequence of NG-ABE8
SEQ ID NO: 9
MKRTADGSEFESPKKKRKVSEVEFSHEYWMRHALTLAKRARDEREVPVGA
VLVLNNRVIGEGWNRAIGLHDPTAHAEIMALRQGGLVMQNYRLIDATLYV
TFEPCVMCAGAMIHSRIGRVVFGVRNSKRGAAGSLMNVLNYPGMNHRVEI
TEGILADECAALLCDFYRMPRQVFNAQKKAQSSINSGGSSGGSSGSETPG
TSESATPESSGGSSGGSDKKYSIGLAIGTNSVGWAVITDEYKVPSKKFKV
LGNTDRHSIKKNLIGALLEDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNEDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDELDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEVYGDYK
VYDVRKMIAKSEQEEITLANGEIRKRPLIETNGETGEIVWDKGRDFATVR
KVLSMPQVNIVKKTEVQTGGFSKESIRPKRNSDKLIARKKDWDPKKYGGF
VSPTVAYSVLVVAKVEKGKSKKLKSVKELLGITIMERSSFEKNPIDFLEA
KGYKEVKKDLIIKLPKYSLFELENGRKRMLASARFLQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPRAFKYFDTTIDRK
VYRSTKEVLDATLIHQSITGLYETRIDLSQLGGDSGGSKRTADGSEFEPK
KKRKSGGSKRTADGSEFEPKKKRK*

The second component of the gene-editing platform disclosed herein consists of at least one guide RNA molecule suitable for guiding the base-editor to at least one target sequence that comprises the IVS2-1 (G>A) mutation. The guide RNA molecule of the present invention thus comprises a guide sequence for providing the targeting specificity. It includes a region that is complementary and capable of hybridization to a pre-selected target site of interest.

In some embodiment, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In some embodiments, the guide sequence is about 17-20 nucleotides in length, such as 20 nucleotides.

Typically, a software program is used to identify candidate CRISPR target sequences on both strands of the DNA nucleic acid molecule based on desired guide sequence length and a CRISPR motif sequence (PAM) for a specified CRISPR enzyme. One requirement for selecting a suitable target nucleic acid is that it has a 3′ PAM site/sequence. Each target sequence and its corresponding PAM site/sequence are referred herein as a Cas-targeted site. Type II CRISPR system, one of the most well characterized systems, needs only Cas 9 protein and a guide RNA complementary to a target sequence to affect target cleavage. For example, target sites for Cas9 from S. pyogenes, with PAM sequences NGG, may be identified by searching for 5′-Nx-NGG-3′ both on the input sequence and on the reverse-complement of the input. Since multiple occurrences in the genome of the DNA target site may lead to nonspecific genome editing, after identifying all potential sites, the program filters out sequences based on the number of times they appear in the relevant reference genome. For those CRISPR enzymes for which sequence specificity is determined by a “seed” sequence, such as the 11-12 bp 5′ from the PAM sequence, including the PAM sequence itself, the filtering step may be based on the seed sequence. Thus, to avoid editing at additional genomic loci, results are filtered based on the number of occurrences of the seed:PAM sequence in the relevant genome. The user may be allowed to choose the length of the seed sequence. The user may also be allowed to specify the number of occurrences of the seed:PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s). Further details of methods and algorithms to optimize sequence selection can be found in U.S. application Ser. No. 61/836,080; incorporated herein by reference.

In some embodiments, the guide RNA targets a sequence selected from Table 1 (see EXAMPLE).

In some embodiments, the gene editing platform comprises a) the adenine base-editor NG-ABE8e or SpRY-ABE8e and b) and at least one gRNA molecule that targets a sequence selected from Table 1.

The guide RNA molecule of the present invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U). In particular, the RNA molecule of the present invention can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties.

In some embodiments, the guide RNA molecule may include one or more modifications. Such modifications may include inclusion of at least one non-naturally occurring nucleotide, or a modified nucleotide, or analogs thereof. Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine.

In some embodiments, the different components of the gene editing platform of the present invention are provided to the eukaryotic cell through expression from one or more expression vectors. For example, the nucleic acids encoding the guide RNA molecule or the base-editor can be cloned into one or more vectors for introducing them into the eukaryotic cell. The vectors are typically prokaryotic vectors, e.g., plasmids, or shuttle vectors, or insect vectors, for storage or manipulation of the nucleic acid encoding the guide RNA molecule or the base-editor herein disclosed. Preferably, the nucleic acids are isolated and/or purified. Thus, the present invention provides recombinant constructs or vectors having sequences encoding one or more of the guide RNA molecule or base-editors described above. Examples of the constructs include a vector, such as a plasmid or viral vector, into which a nucleic acid sequence of the invention has been inserted, in a forward or reverse orientation. In some embodiments, the construct further includes regulatory sequences. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the eukaryotic cell to be transformed, transfected, or infected, the desired expression level, and the like. Large numbers of suitable vectors and promoters are known to those of skill in the art, and are commercially available. Appropriate cloning and expression vectors for use with eukaryotic hosts are also described in e.g., Sambrook et al. (2001, Molecular Cloning: A Laboratory Manual, Cold Spring Harbor Press). The vector can be capable of autonomous replication or integration into a host DNA. The vector may also include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli. Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

In some embodiments, the different components of the gene editing platform of the present invention are provided to the population of cells through the use of an RNA-encoded system. In particular, the base-editing system may be provided to the population of cells through the use of a chemically modified mRNA-encoded adenine or cytidine base editor together with modified guide RNA as described in Jiang, T., Henderson, J. M., Coote, K. et al. Chemical modifications of adenine base editor mRNA and guide RNA expand its application scope. Nat Commun 11, 1979 (2020). In particular, engineered RNA-encoded base-editors (e.g. ABE) system are prepared by introducing various chemical modifications to both mRNA that encoded the base-editor and guide RNA. In particular said modifications consist in uridine depleted mRNAs modified with 5-methoxyuridine: synonymous codons may be introduced to deplete uridines as much as possible without altering the coding sequence and replaced all the remaining uridines with 5-methoxyuridine. Said optimized base editing system exhibits higher editing efficiency at some genomic sites compared to DNA-encoded system. It is also possible to encapsulate the modified mRNA and guide RNA into lipid nanoparticle (LNP) for allowing lipid nanoparticle (LNP)-mediated delivery.

In some embodiments, the different components of the gene editing platform of the present invention are provided to the population of cells through the use of ribonucleoprotein (RNP) complexes. For instance, the base-editor can be pre-complexed with one or more guide RNA molecules to form a ribonucleoprotein (RNP) complex. The RNP complex can thus be introduced into the eukaryotic cell. Introduction of the RNP complex can be timed. The cell can be synchronized with other cells at G1, S, and/or M phases of the cell cycle. RNP delivery avoids many of the pitfalls associated with mRNA, DNA, or viral delivery. Typically, the RNP complex is produced simply by mixing the proteins (i.e. the base-editor) and one or more guide RNA molecules in an appropriate buffer. This mixture is incubated for 5-10 min at room temperature before electroporation. Electroporation is a delivery technique in which an electrical field is applied to one or more cells in order to increase the permeability of the cell membrane. In some embodiments, genome editing efficiency can be improved by adding a transfection enhancer oligonucleotide.

In some embodiments, a plurality of successive transfections are performed for reaching a desired level of mutagenesis in the cell.

A further object of the present invention relates to a method of treating β-thalassemia in a subject in need thereof, the method comprising transplanting a therapeutically effective amount of a population of eukaryotic cells obtained by the method as above described.

In some embodiments, the population of eukaryotic cells is autologous to the subject, meaning the population of cells is derived from the same subject.

In some embodiments, the patient suffers from sickle β-thalassemia.

### Kits

This invention further provides kits containing reagents for performing the above-described methods, including all component of the gene editing platform as disclosed herein for performing mutagenesis. To that end, one or more of the reaction components, e.g., guide RNA molecules, and nucleic acid molecules encoding for the base-editors for the methods disclosed herein can be supplied in the form of a kit for use. In some embodiments, the kit comprises one or more base-editors and one or more guide RNA molecules. In some embodiments, the kit can include one or more other reaction components. In some embodiments, an appropriate amount of one or more reaction components is provided in one or more containers or held on a substrate. Examples of additional components of the kits include, but are not limited to, one or more host cells, one or more reagents for introducing foreign nucleotide sequences into host cells, one or more reagents (e.g., probes or PCR primers) for detecting expression of the guide RNA or base-editors or verifying the target nucleic acid's status, and buffers or culture media for the reactions. The kit may also include one or more of the following components: supports, terminating, modifying or digestion reagents, osmolytes, and an apparatus for detection. The components used can be provided in a variety of forms. For example, the components (e.g., enzymes, RNAs, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C., preferably at or below −20° C., or otherwise in a frozen state. The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

The invention will be further illustrated by the following figures and examples. However, these examples and figures should not be interpreted in any way as limiting the scope of the present invention.

### FIGURES

FIG. 1. Design and screening of gRNAs targeting the IVS2-1 (G>A) mutation in β-thalassemic T cells.

A. gRNAs1-3 were manually designed to place the IVS2-1 (G>A) mutation in position 6 to 8 of the editing window. The mutation is highlighted with a grey box. B. Overview of the cell collection for testing the ability of gRNA/BE to revert the IVS2-1 (G>A) mutation. Peripheral blood mononuclear cells (PBMCs) were isolated from an homozygous beta thalassemia patient harboring the IVS2-1 (G>A) mutation. After CD34⁺ cell sorting, T cells were recovered from the negative fraction for testing gRNA/BE combinations, before moving to CD34⁺ cells with a selected strategy. C. Frequency of corrected alleles (normalized to the frequency of GFP⁺ cells) as evaluated by EditR and InDel frequency as assessed by TIDE in T cells transfected with different combinations of synthetic gRNAs and ABE mRNAs. Data are expressed as mean±standard error of the mean (SEM) (n=3 biologically independent experiments, 1 homozygous donor). D. Representative percent composition of Sanger sequencing traces measured to be significantly different from noise (in grey), as assessed by EditR following Sanger sequencing in T cells transfected with gRNA2/SpRY-ABE8e. Target base position is highlighted with a black box and the bystander edits with grey boxes (b0 and b1).

FIG. 2. Efficient correction of the IVS2-1 (G>A) mutation in β-thalassemic HSPCs.

A. Experimental protocol used for base editing experiments in β-thalassemic HSPCs. SpRY-ABE8e mRNA and synthetic gRNA2 were co-transfected in β-thalassemic HSPCs. Cells were differentiated into mature RBCs using a three-phase erythroid differentiation protocol. B. Frequency of corrected alleles as measured by targeted NGS sequencing and InDel frequency in corrected β-thalassemic samples. Data are expressed as mean±SEM (n=2 biologically independent experiments, 1 donor). C. Frequency and sequence of modified and unmodified alleles in corrected β-thalassemic samples, as measured by targeted NGS sequencing. Target base position is highlighted with a bold black box. Bystander edits are present at positions 4 and 10 (grey boxes, b0 and b1).

FIG. 3. Off-target editing in β-thalassemic cells.

Frequency of base editing (A) and InDels (B) at the 10 predicted off-targets (OTs) in control (BT-TE) and edited (BT-cor) β-thalassemic samples, as measured by targeted NGS sequencing (n=3 biologically independent experiments, 2 donors). Off-target sequences are reported in Table 3. *p<0.05, **p<0.001 (Multiple paired t-tests).

FIG. 4. Efficient reversion of the IVS2-1 (G>A) mutation in β-thalassemic HSPCs corrects globin and hemoglobin expression.

A. RT-PCR using primers amplifying a region spanning the HBB exon 1-2 and exon 2-3 junctions. cDNA was obtained from erythroid cells derived from corrected BT HSPCs (cor). As controls, we used erythroid cells derived from BT or HD HSPCs electroporated with ABE mRNA only (3 HDs, 1 BT, 2 independent replicates). B. Relative quantification of β-globin mRNAs as evaluated by long-read sequencing of the β-globin transcriptome. cDNA was obtained from erythroid cells derived from corrected BT HSPCs (cor). As controls, we used erythroid cells derived from BT or HD HSPCs electroporated with ABE mRNA only (3 HDs, 1 BT, 2 independent replicates). C. RT-qPCR using primers detecting wild-type β-globin mRNAs in erythroid cells derived from corrected β-thalassemic HSPCs (cor). β-globin expression was normalized to α-globin. Data are expressed as mean±SEM. As controls, we used erythroid cells derived from β-thalassemic patient's or healthy donor HSPCs transfected only with SpRY-ABE8e mRNA (BE). Dotted lines indicate maximum and minimum values observed in HD cells. D. RT-qPCR using primers detecting γ-globin mRNAs in erythroid cells derived from corrected β-thalassemic HSPCs (cor). γ-globin expression was normalized to α-globin. Data are expressed as mean±SEM. As controls, we used erythroid cells derived from β-thalassemic patient's or healthy donor HSPCs transfected only with SpRY-ABE8e mRNA (BE). E. Expression of β-, β Nîmes-, Gγ-, Aγ- and δ-globin chains measured by RP-HPLC in β-thalassemic patient and healthy donor RBCs. β-like-globin expression was normalized to α-globin. The ratio α/non-α globins is reported on top of the graph. RBCs were obtained from corrected β-thalassemic HSPCs (cor). As controls, we used RBCs derived from β-thalassemic patient's or healthy donor HSPCs transfected with TE (TE) or only with SpRY-ABE8e mRNA (BE) (n=2 biologically independent experiments, 1 β-thalassemia patient and 2 healthy donors). Data are expressed as mean±SEM. F. Analysis of HbA, Hb-Nîmes, HbF and HbA₂ by CE-HPLC in β-thalassemic patient and healthy donor RBCs. We calculated the percentage of each Hb type over the total Hb tetramers. RBCs were obtained from corrected β-thalassemic HSPCs (cor). As controls, we used RBCs derived from β-thalassemic patients' or healthy donor HSPCs transfected with TE (TE) or only with SpRY-ABE8e mRNA (BE) (n=2 biologically independent experiments, 1 β-thalassemia patient and 2 healthy donors). Data are expressed as mean±SEM. BT, β-thalassemia patients. HD, healthy donors.

FIG. 5. Efficient reversion of the IVS2-1 (G>A) mutation in β-thalassemic HSPCs corrects ineffective erythropoiesis.

A-C. Frequency of CD36⁺ (A), CD71⁺ (B) and GPA⁺ (C) cells at day 13, 16 and 19 of erythroid differentiation, as measured by flow cytometry analysis. As controls, we used RBCs derived from β-thalassemic patient's or healthy donor HSPCs transfected with TE only (TE) or with SpRY-ABE8e mRNA only (BE) (n=2 biologically independent experiments, 1 β-thalassemia patient and 3 healthy donors). Samples that were simultaneously differentiated are indicated with the same symbol (∇: differentiation 1, □: differentiation 2). Data are expressed as mean±SEM. D. Frequency of enucleated cells at day 13, 16 and 19 of erythroid differentiation, as measured by flow cytometry analysis of cells stained with the DRAQ5 nuclear dye. As controls, we used RBCs derived from β-thalassemic patient's or healthy donor HSPCs transfected with TE or only with SpRY-ABE8e mRNA (ctr) (n=2 biologically independent experiments, 1 β-thalassemia patient and 3 healthy donors). Samples that were simultaneously differentiated are indicated with the same symbol (▾: differentiation 1, ▪: differentiation 2). Data for HD samples are expressed as mean±SEM. E. Cell size of enucleated cells at day 13, 16 and 19 of erythroid differentiation, as measured by flow cytometry using the median of FSC-A intensity. As controls, we used RBCs derived from β-thalassemic patient's or healthy donor HSPCs transfected with TE or only with SpRY-ABE8e mRNA (ctr) (n=2 biologically independent experiments, 1 β-thalassemia patient and 3 healthy donors). Samples that were simultaneously differentiated are indicated with the same symbol (▾: differentiation 1, ▪: differentiation 2). Data for HD samples are expressed as mean±SEM. F. Flow cytometry histograms showing the frequency of apoptotic cells (AnnexinV⁺-cells) in the 7AAD⁻ cell population in unstained (Uns), β-thalassemic and healthy donor samples at day 13 of erythroid differentiation. As controls, we used RBCs derived from β-thalassemic patient's or healthy donor HSPCs transfected with SpRY-ABE8e mRNA only (BE) (n=2 biologically independent experiments, 1 β-thalassemia patient and 3 healthy donors).

### EXAMPLE

### Material & Methods

### HSPC and T Cell Purification and Culture

We obtained human non-mobilized peripheral blood CD34⁺ HSPCs from β-thalassemia patients. Samples eligible for research purposes were obtained from the “Hôpital Necker-Enfants malades” Hospital (Paris, France). Written informed consent was obtained from all adult subjects. All experiments were performed in accordance with the Declaration of Helsinki. The study was approved by the regional investigational review board (reference: DC 2014-2272, CPP Ile-de-France II “Hôpital Necker-Enfants malades”). HSPCs were purified by immunomagnetic selection immunostaining with the CD34 MicroBead Kit (Miltenyi Biotec). The CD34⁻ fraction was kept for T cell cultures. Forty-eight hours before transfection, CD34⁺ cells were thawed and cultured at a concentration of 5×10⁵ cells/mL in the “HSPC medium” containing StemSpan (STEMCELL Technologies) supplemented with penicillin/streptomycin (Gibco), 250 nM StemRegenin1 (STEMCELL Technologies), and the following recombinant human cytokines (PeproTech): human stem cell factor (SCF) (300 ng/ml), Flt-3L (300 ng/ml), thrombopoietin (TPO) (100 ng/ml), and interleukin-3 (IL-3) (60 ng/ml). Four days before transfection, the CD34⁻ fraction was thawed and cultured at 5×10⁶ cells/mL in the “T cells medium” containing RPMI 1640+GlutaMAX (Gibco) supplemented with FBS (Thermo), penicillin/streptomycin (Gibco) and Recombinant Human IL-2 (Peprotech). After recovery, cells were transferred to “T cell activation medium” supplemented with CD28 Monoclonal Antibody (eBioscience, Clone CD28.2) in plates coated with CD3 Monoclonal Antibody (eBioscience, Clone OKT3).

### Base Editor Plasmids

Constructs used in this study include NG-ABE8e (Plasmid #138491, Addgene) and SpRY-ABE8e. The SpRY-ABE8e plasmid was created by replacing the Cas9 coding sequence of the ABE8e plasmid (Plasmid #138489, Addgene)¹⁹ with the Cas9 fused to GFP included in the “pCMV-T7-ABEmax (7.10)-SpRY-P2A-EGFP (RTW5025)” plasmid (Plasmid #140003, Addgene)²⁰.

gRNA Design

We manually designed gRNAs targeting the IVS2-1 (G>A) mutation (Table 1). We used chemically modified synthetic gRNAs harboring 2′-O-methyl analogs and 3′-phosphorothioate nonhydrolyzable linkages at the first three 5′ and 3′ nucleotides (Synthego).

TABLE 1
gRNA target sequences.

| | | PAM | | | |
|---|---|---|---|---|---|
| | | sequence | | | Compatible |
| gRNA | Sequence (5′ to 3′) | (5′ to 3′) | Position (hg19) | Strand | ABEs |
| gRNA1 | TCAGGATGAGTCTATGGGAC | GCTT | chr11: | + | SpRY- |
| | (SEQ ID NO: 10) | | 5247792-5247811 | | ABE8e |
| | | | (HBB) | | |
| gRNA2 | TTCAGGATGAGTCTATGGGA | CGCT | chr11: | + | NG- |
| | (SEQ ID NO: 11) | | 5247793-5248017 | | ABE8e, |
| | | | 5247812 | | SpRY- |
| | | | (HBB) | | ABE8e |
| gRNA3 | CTTCAGGATGAGTCTATGGG | ACGC | chr11: | + | SpRY- |
| | (SEQ ID NO: 12) | | 5247794-5247813 | | ABE8e |
| | | | (HBB) | | |

mRNA In Vitro Transcription

20 μg of NG-ABE8e or SpRY-ABE8e expressing plasmids were digested overnight with SapI restriction enzyme (Thermo) that cleaves once right after the poly-A tail. The linearized plasmids were purified using a PCR purification kit (QIAGEN #28106) and were eluted in 14 μl of DNase/RNase-free water. 2 μg of linearized plasmid were used as template for the in vitro transcription reaction (MEGAscript, Ambion #AM1334). The in vitro transcription protocol was modified as follows. The GTP nucleotide solution was used at a final concentration of 3.0 mM instead of 7.5 mM and the anti-reverse cap analog N7-Methyl-3′-O-Methyl-Guanosine-5′-Triphosphate-5′-Guanosine (ARCA, Trilink #N-7003) was used at a final concentration of 12.0 mM resulting in a final ratio of Cap:GTP of 4:1 that allows efficient mRNA capping. The incubation time for the in vitro reaction was reduced to 30 minutes. mRNA was precipitated using lithium chloride and resuspended in TE buffer in a final volume that allowed to achieve a concentration of >1 μg/μl. The mRNA quality was assessed using Bioanalyzer (Agilent).

### RNA Transfection

1×10⁶ T cells per condition were transfected with 3.0 μg of the ABE-encoding mRNA and 3.2 μg of the synthetic gRNA. When ABE was not fused to GFP, a GFP-encoding mRNA (Tebu-bio) was added to the transfection mix. We used the P3 Primary Cell 4D-Nucleofector X Kit S (Lonza) and the EO115 program (Nucleofector 4D). Cells transfected only with TE buffer served as negative controls.

1×10⁴ to 5×10⁵ HSPCs per condition were transfected with 3.0 μg of the ABE-encoding mRNA and 3.2 μg of the synthetic gRNA. We used the P3 Primary Cell 4D-Nucleofector X Kit S (Lonza) and the CA137 program (Nucleofector 4D). Cells transfected only with TE buffer or only with the ABE-encoding mRNA served as negative controls.

### HSPC Differentiation

Transfected CD34⁺ HSPCs were differentiated into mature red blood cells (RBCs) using a three-phase erythroid differentiation protocol, as previously described^{21,22}. During the first phase (day 0 to day 6), cells were cultured in a basal erythroid medium supplemented with 100 ng/ml recombinant human SCF (PeproTech), 5 ng/ml recombinant human IL-3 (PeproTech), 3 IU/ml EPO Eprex (Janssen-Cilag) and 10⁻⁶ M hydrocortisone (Sigma). During the second phase (day 6 to day 9), cells were co-cultured with MS-5 stromal cells in the basal erythroid medium supplemented with 3 IU/ml EPO Eprex (Janssen-Cilag). During the third phase (day 9 to day 20), cells were co-cultured with stromal MS-5 cells in a basal erythroid medium without cytokines. Erythroid differentiation was monitored by flow cytometry analysis of CD36, CD71, GYPA and of enucleated cells using the DRAQ5 double-stranded DNA dye. 7AAD was used to identify live cells.

### Evaluation of Editing Efficiency

Genomic DNA was extracted from control and edited cells using PURE LINK Genomic DNA Mini kit (LifeTechnologies), or Quick-DNA/RNA Miniprep (ZYMO Research), following manufacturers' instructions. To evaluate base editing efficiency at gRNA target sites, we performed a nested PCR using previously published primers²³, followed by Sanger sequencing and EditR analysis²⁴. TIDE analysis (Tracking of InDels by Decomposition) was performed to evaluate the percentage of InDels in edited samples²⁵.

On- and off-target regions in HSPC-derived erythroid cells were also PCR-amplified and subjected to NGS. Off-targets were in silico predicted using COSMID²⁶. We assessed editing at day 9 or 13 of differentiation. On-target and off-target sites were PCR-amplified using the Phusion High-Fidelity polymerase (NEB, M0530) and primers containing specific DNA stretches (MR3 for forward primers and MR4 for reverse primers; Table 2). Amplicons were purified using Ampure XP beads (Beckman Coulter, A63881). Illumina-compatible barcoded DNA amplicon libraries were prepared by a second PCR step using the Phusion High-Fidelity polymerase (NEB, M0530) and primers containing Unique Dual Index (UDI) barcodes and annealing to MR3 and MR4 sequences. Libraries were pooled, purified using the High Pure PCR Product Purification Kit (Sigma-Aldrich, 11732676001), and sequenced using Illumina NovaSeq 6000 system (paired-end sequencing; 2×100-bp) to obtain a minimum of 100,000 reads per amplicon. Targeted NGS data were analyzed using CRISPResso2²⁷.

TABLE 2
PCR primers to amplify on-target and off-target sites

| Amplified | | |
|---|---|---|
| region | F/R | Sequence (5′ to 3′) |
| On target | F | GCAGCGTCAGATGTGTATAAGAGACAGGTACCCTGTTACTTATCCCCT |
| | | TCC (SEQ ID NO: 14) |
| | R | TGGGCTCGGAGATGTGTATAAGAGACAGCTCAAGGGCACCTTTGCCAC |
| | | (SEQ ID NO: 15) |
| OT1 | F | GCAGCGTCAGATGTGTATAAGAGACAGAGTCCTAGTCAGAGAAATTGG |
| | | GC (SEQ ID NO: 16) |
| | R | TGGGCTCGGAGATGTGTATAAGAGACAGAGTCATTTTAATCCAGTCCA |
| | | GAAGT (SEQ ID NO: 17) |
| OT2 | F | GCAGCGTCAGATGTGTATAAGAGACAGCTGTGTTGAATGCTTTCTGGT |
| | | GGC (SEQ ID NO: 18) |
| | R | TGGGCTCGGAGATGTGTATAAGAGACAGGACCAATTGGTCAGAGGCCA |
| | | AG (SEQ ID NO: 19) |
| OT3 | F | GCAGCGTCAGATGTGTATAAGAGACAGTACAGGGAAGTCCGTTTCACT |
| | | CAG (SEQ ID NO: 20) |
| | R | TGGGCTCGGAGATGTGTATAAGAGACAGACGTGGCTGTGTACAAAAGT |
| | | CTGC (SEQ ID NO: 21) |
| OT4 | F | GCAGCGTCAGATGTGTATAAGAGACAGAACTCTCCAAAAAGGCAGCAG |
| | | TAAAACAGC (SEQ ID NO: 22) |
| | R | TGGGCTCGGAGATGTGTATAAGAGACAGTTCCCTGTAACTTGGCAAGG |
| | | AGC (SEQ ID NO: 23) |
| OT5 | F | GCAGCGTCAGATGTGTATAAGAGACAGATGCCTCAGTACAGTACCCCT |
| | | GG (SEQ ID NO: 24) |
| | R | TGGGCTCGGAGATGTGTATAAGAGACAGGCTGTGGAGGAAAAAGACAC |
| | | TGC (SEQ ID NO: 25) |
| OT6 | F | GCAGCGTCAGATGTGTATAAGAGACAGATGTGTCATTTTGGCTGGGC |
| | | (SEQ ID NO: 26) |
| | R | TGGGCTCGGAGATGTGTATAAGAGACAGGCCTTTTGTTGTAGTCAGGG |
| | | C (SEQ ID NO: 27) |
| OT7 | F | GCAGCGTCAGATGTGTATAAGAGACAGTGATGAGGCCAAGGACCTGGT |
| | | T (SEQ ID NO: 28) |
| | R | TGGGCTCGGAGATGTGTATAAGAGACAGGAGCTGTGAAAGCAACCGTA |
| | | CACT (SEQ ID NO: 29) |
| OT8 | F | GCAGCGTCAGATGTGTATAAGAGACAGCCTACAGCTGCCTATATGTTA |
| | | CA (SEQ ID NO: 30) |
| | R | TGGGCTCGGAGATGTGTATAAGAGACAGTCCTTTTCTTCCGCATCCTT |
| | | G (SEQ ID NO: 31) |
| OT9 | F | GCAGCGTCAGATGTGTATAAGAGACAGCCTTGTGATTCTCCCATTGGA |
| | | GC (SEQ ID NO: 32) |
| | R | TGGGCTCGGAGATGTGTATAAGAGACAGCCAGTTTTCAGTGGGATCCA |
| | | TAGC (SEQ ID NO: 33) |
| OT10 | F | GCAGCGTCAGATGTGTATAAGAGACAGGTTGAACTCCAAAGTGCAGAA |
| | | GGC (SEQ ID NO: 34) |
| | R | TGGGCTCGGAGATGTGTATAAGAGACAGCCTGGTGCCTGTAATTATAC |
| | | CAGC (SEQ ID NO: 35) |

### Flow Cytometry Analysis

Flow cytometry analysis of CD36, CD71 and GYPA erythroid surface markers on HSPC-derived erythroid cells was performed using a V450-conjugated anti-CD36 antibody (561535, BD Horizon), a FITC-conjugated anti-CD71 antibody (555536, BD Pharmingen) and a PE-Cy7-conjugated anti-GYPA antibody (563666, BD Pharmingen). Flow cytometry analysis of enucleated or viable cells was performed using double-stranded DNA dyes (DRAQ5, 65-0880-96, Invitrogen and 7AAD, 559925, BD, respectively). Apoptosis was evaluated using PE Annexin V Apoptosis Detection Kit I (BD Biosciences). Flow cytometry analyses were performed using Gallios (Beckman coulter) flow cytometer. Data were analyzed using the FlowJo (BD Biosciences) software.

### RT-PCR, RT-qPCR and β-Globin Transcriptome Analysis

RNA was extracted from cells at day 13 of differentiation (Qiagen, 74004 or Zymo Research, ZD7001) and retro-transcribed (Thermo, 18080051). RT-PCR analysis of β-globin mRNAs was performed using previously described primers spanning exon 1 to 2 and exon 2 to 3 junctions²⁸. RT-qPCR was performed using previously described primers specifically detecting β-globin mRNA correctly spliced at the exon 2 to 3 junction²³ and the following primers amplifying γ-globin and α-globin cDNAs, respectively: γ-globin-F 5′-CCTGTCCTCTGCCTCTGCC-3′ (SEQ ID NO: 36), γ-globin-R 5′-GGATTGCCAAAACGGTCAC-3′ (SEQ ID NO: 37), α-globin-F 5′-CGGTCAACTTCAAGCTCCTAA-3′ (SEQ ID NO: 38) and α-globin-R 5′-ACAGAAGCCAGGAACTTGTC-3′ (SEQ ID NO: 39). The β-globin transcriptome was amplified and sequenced on MinION device.

### RP-HPLC Analysis of Globin Chains

Reversed-phase HPLC analysis was performed using a NexeraX2 SIL-30AC chromatograph and the LC Solution software (Shimadzu). A 250×4.6 mm, 3.6 μm Aeris Widepore column (Phenomenex) was used to separate globin chains by HPLC. Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm.

### CE-HPLC Analysis of Hemoglobin Tetramers

Cation-exchange HPLC analysis was performed using a NexeraX2 SIL-30AC chromatograph and the LC Solution software (Shimadzu). A 2 cation-exchange column (PolyCAT A, PolyLC, Columbia, MD) was used to separate hemoglobin tetramers by HPLC. Samples were eluted with a gradient mixture of solution A (20 mM bis Tris, 2 mM KCN, pH=6.5) and solution B (20 mM bis Tris, 2 mM KCN, 250 mM NaCl, pH-6.8). The absorbance was measured at 415 nm.

### Results

### Adenine Base Editing is an Efficient Tool to Revert the IVS2-1 (G>A) Mutation

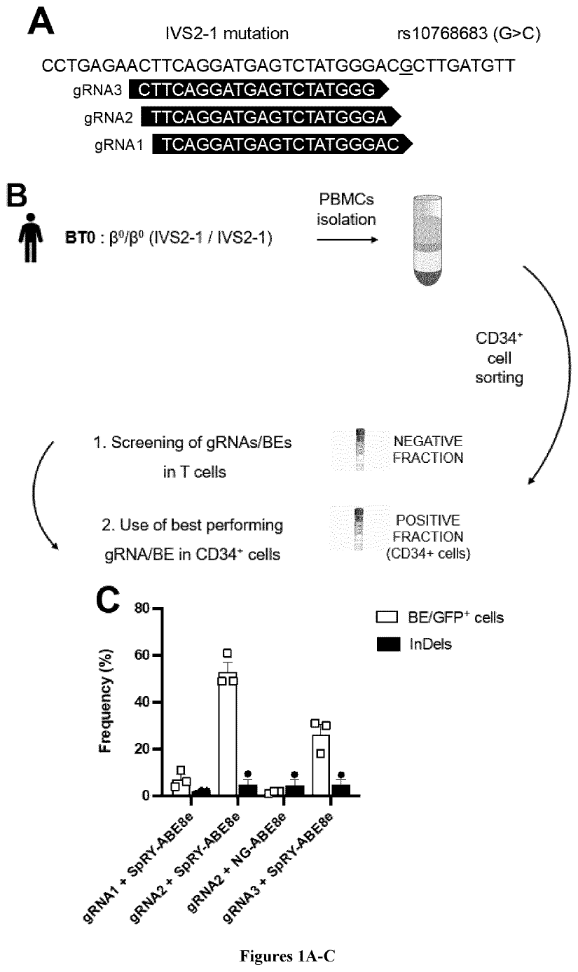

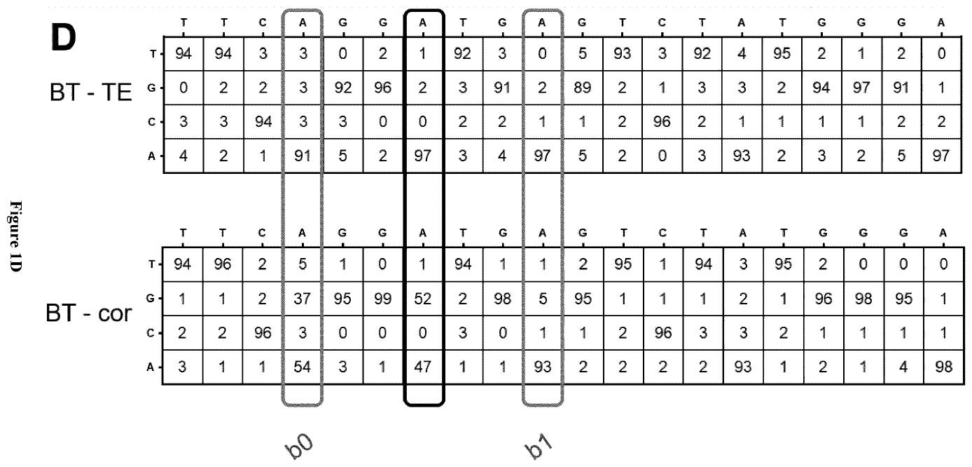

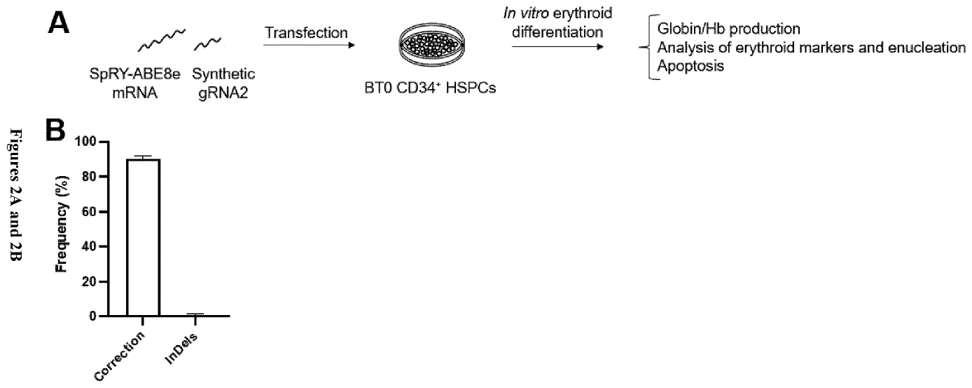

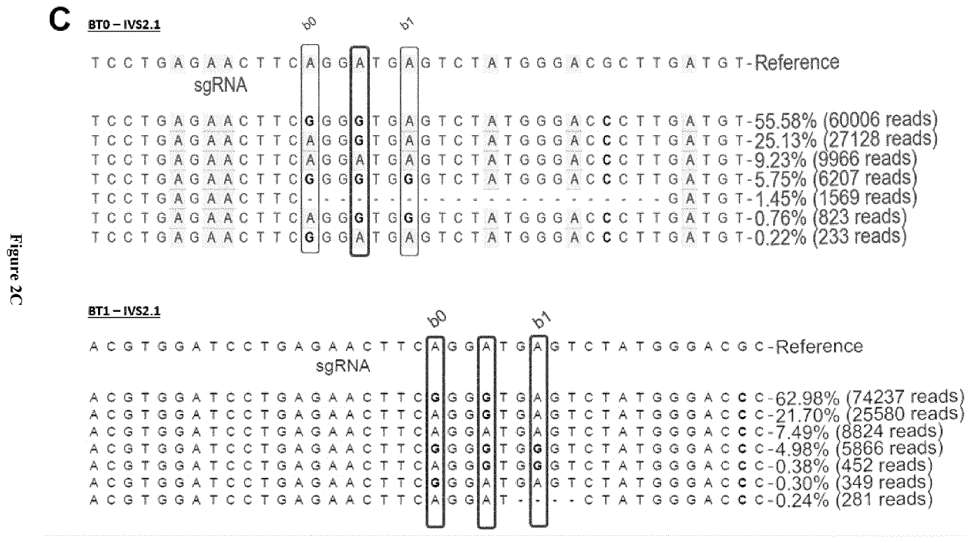

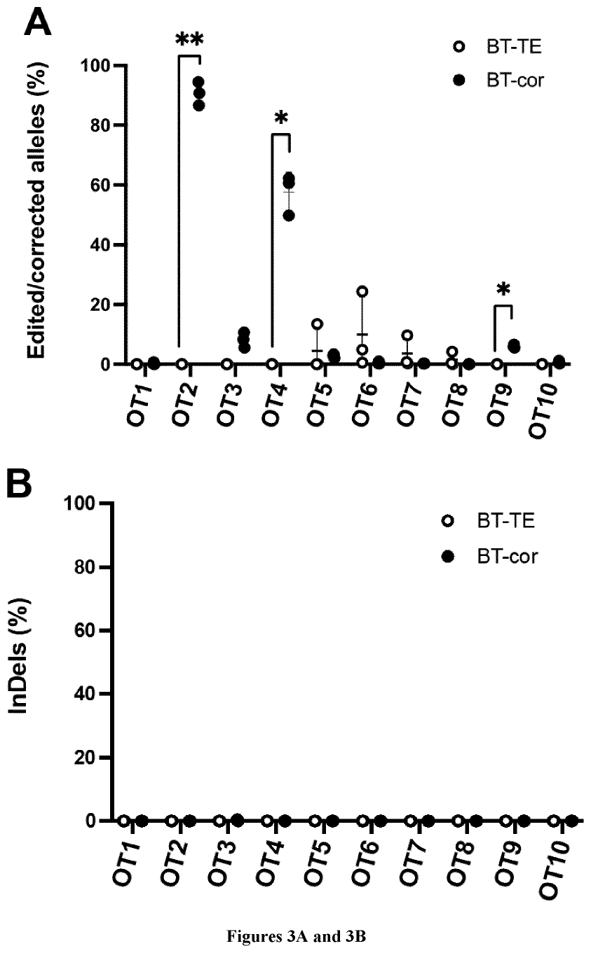

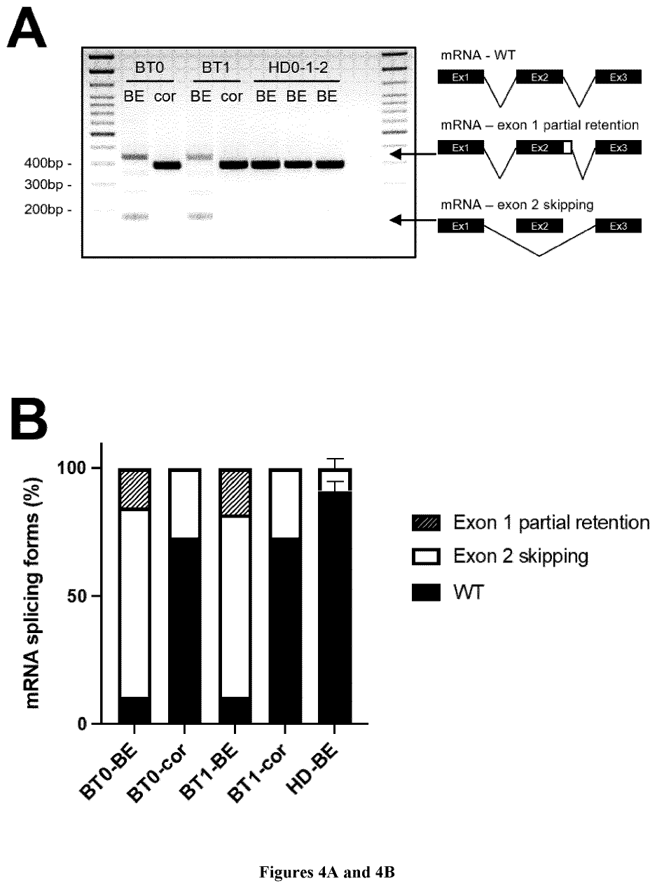

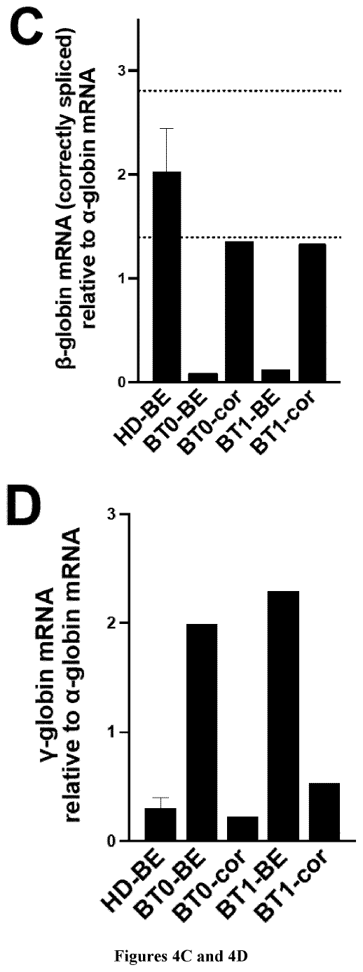

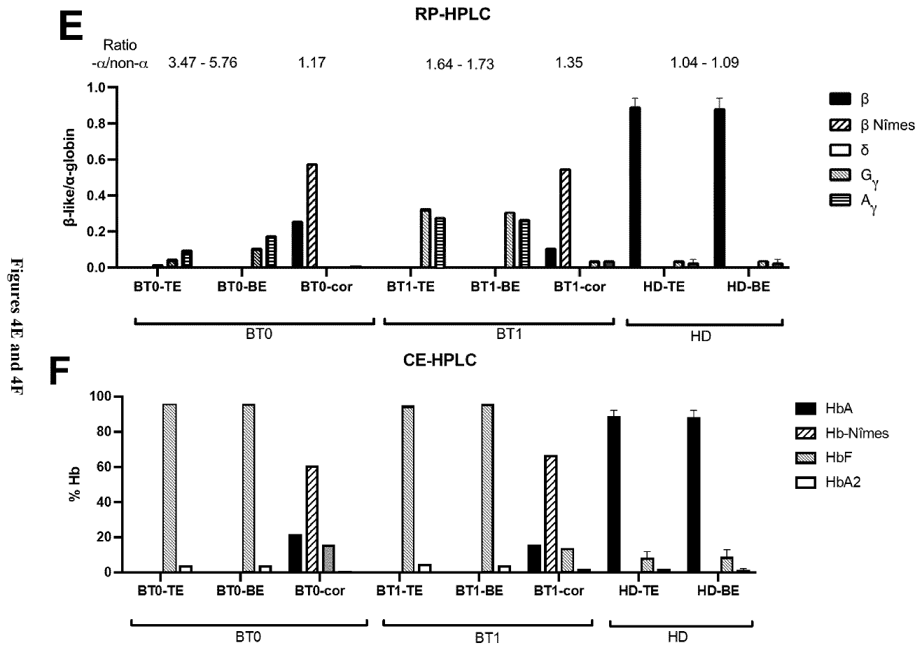

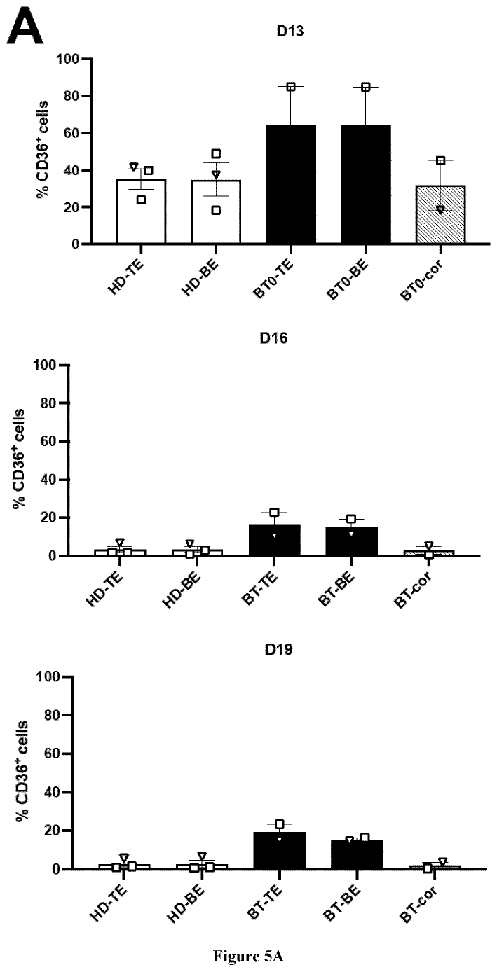

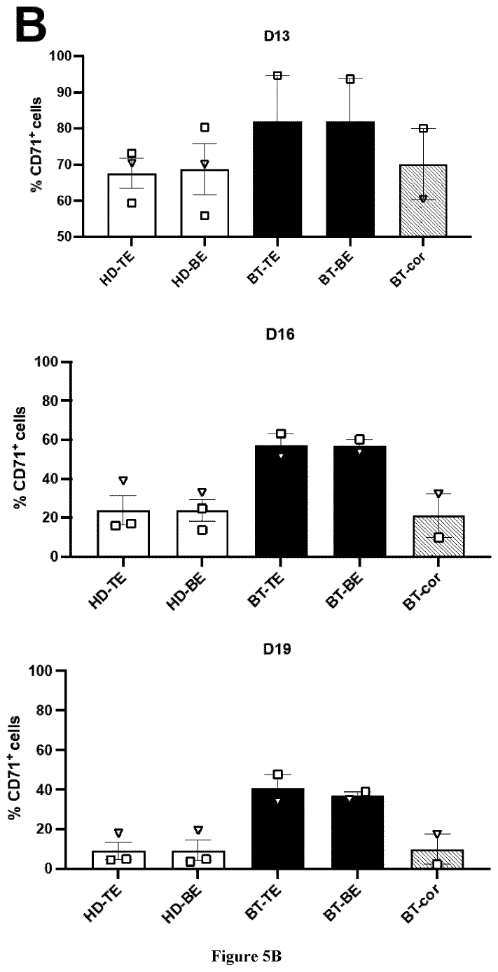

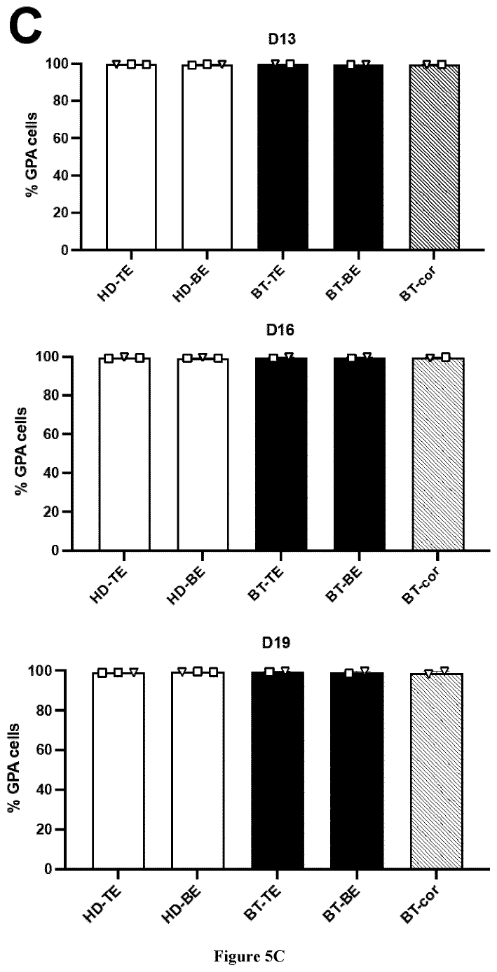

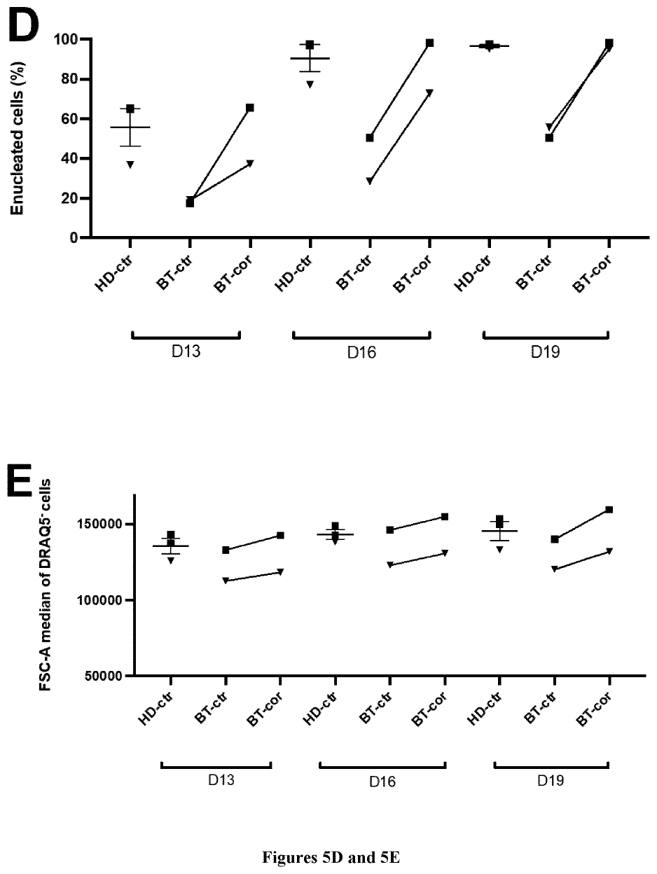

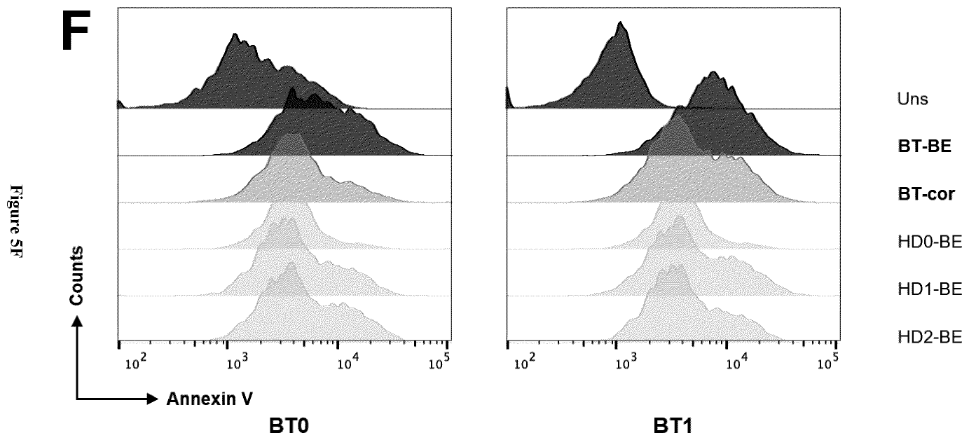

ABEs allow A>G conversions and can potentially correct the IVS2-1 (G>A) mutation. In particular, we used NG-ABE8e and SpRY-ABE8e²⁹ (Table 1). The latter was generated by combining the highly processive deaminase from ABE8e¹⁹ with the PAM-less Cas9 nickase SpRY²⁰ (SpRY-ABE8e). This combination allowed the design of three gRNAs (1 to 3) placing the target base within positions 6 to 8 of the canonical editing window (FIG. 1A). Of note, these gRNAs were designed in order to avoid overlapping a common SNP located near the IVS2-1 mutation³⁰ (FIG. 1A, rs10768683).

We screened gRNA/BE combinations in T cells obtained from a β-thalassemic patient homozygous for the IVS2-1 (G>A) mutation (BT0 patient, FIG. 1B). Cells were transfected with chemically modified gRNAs and in vitro transcribed ABE mRNAs. gRNA2/SpRY-ABE8e was the most efficient combination able to revert the IVS2-1 (G>A) mutation in more than 50% of HBB alleles, as evaluated by Sanger sequencing and EditR analysis (FIG. 1C-D) and was then used for following experiments. Of note, two additional modifications on adjacent adenines were observed in the target sequence (bystander edits) (FIG. 1D, b0 and b1). The first event leads to a missense mutation in the last codon of HBB exon 2, replacing an arginine by a glycine (FIG. 1D, b0). However, this amino acid change has likely no consequences as it was described in a known Hb variant (Hb Nîmes) which is not associated with any clinical abnormalities³¹. The second event lays in the second intron of HBB, and results in an intronic variant with no anticipated consequences (FIG. 1D, b1). In fact, a single nucleotide polymorphism previously described at this position (rs1468286413) was not associated with any clinical manifestation³².

### Efficient Correction of the IVS2-1 (G>A) Mutation in β-Thalassemic HSPCs

HSPCs from a homozygous β-thalassemia patient were transfected with chemically modified gRNA2 and in vitro transcribed SpRY-ABE8e mRNA (FIGS. 1B, 2A). Deep sequencing of edited samples demonstrated that the correction of the targeted mutation was highly efficient and reproducible among the replicates (IVS2-1:90.4%+1.5) (FIG. 2B, C). Moreover, it confirmed the DSB-free nature of ABEs, as we detected very low InDel frequencies in base-edited samples (FIG. 2B, C).

To evaluate the safety profile of our strategy, we performed NGS of the top 10 in silico predicted off-targets (Table 3). Base editing was observed at OT2, OT4 and OT9 sites, mapping to an intronic, an intergenic and a lncRNA exonic site, respectively (FIG. 3A, Table 3). The intronic OT site (OT2) is located away from donor and acceptor splicing sites, thus off-targeting likely does not affect gene splicing and expression. Importantly, no InDels were detected at off-target sites, thus minimizing the possibility of DSB-induced genomic rearrangements (e.g., large deletions and translocations) (FIG. 3B). Of note, off-target activity could be moderated using more specific ABE variants^{19,33-35}.

Following transfection, β-thalassemic and control HD HSPCs were differentiated towards the erythroid lineage (FIG. 2A). Reverse transcription PCR (RT-PCR) showed restoration of the correct β-globin splicing and a substantial decrease of aberrant transcripts characterized by lower or higher mobility owing to partial intron retention or exon skipping, respectively (FIG. 4A). The relative decrease of aberrant transcripts was further confirmed by long-read sequencing of the β-globin transcriptome (FIG. 4B). After correction, wild-type-β-globin mRNA levels were similar to those observed in HD cells (FIG. 4C). On the contrary, γ-globin mRNA expression, typically elevated in untreated thalassemic samples due to the stress erythropoiesis, was substantially reduced after treatment (FIG. 4D). Importantly, after treatment, erythroid cells exhibited high levels of HbA (β-globin) and Hb-Nîmes (β Nîmes-globin) along with lower α/non-α ratios (FIG. 4E-F).

In conclusion, we were able to efficiently correct the IVS2-1 (G>A) mutation in β-thalassemic HSPCs without causing DSBs, and to restore high levels of therapeutic Hb expression in HSPC-derived RBCs.

TABLE 3
In silico predicted off-targets

| Off- | | Mismatches, | | | | |
|---|---|---|---|---|---|---|
| target | Sequence (5′ to 3′) | Ins/Del | Position (hg19) | Strand | Score | Type |
| OT1 | TTCTGGAGGAGTCTATGGGA | 2 | Chr2: | − | 0.44 | Intergenic |
| | (SEQ ID NO: 40) | | 104214701-104214720 | | | |
| OT2 | TTCTGGATGAGCCTATGGGA | 2 | Chr13: | + | 0.97 | UGGT2 |
| | (SEQ ID NO: 41) | | 96502627-96502646 | | | (Intron) |
| OT3 | TTCAGCAGAGTCTATGGGA | 1, Del 13 | Chr10: | + | 0.99 | ATE1 |
| | (SEQ ID NO: 42) | | 123573489-123573507 | | | (Intron) |
| OT4 | TTCAGGATGAGTTTATGGGA | 1 | Chr9: | − | 1.1 | Intergenic |
| | (SEQ ID NO: 43) | | 85553299-85553318 | | | |
| OT5 | TGCAGGAGTGAGTCTATGGG | 1, Ins 13 | Chr10: | + | 1.1 | PIK3API |
| | A (SEQ ID NO: 44) | | 98398423-98398443 | | | (Intron) |
| OT6 | TTCAGGATGTTTCTATGGGA | 2 | Chr7: | + | 1.2 | Intergenic |
| | (SEQ ID NO: 45) | | 27745834-27745853 | | | |
| OT7 | TTAAGGATGCAGTCTATGGG | 1, Ins 11 | Chr18: | + | 1.35 | DCC |
| | A (SEQ ID NO: 46) | | 50791495-50791515 | | | (Intron) |
| OT8 | TTTAGGATGAGTCGATGGGA | 2 | Chr5: | + | 1.45 | ADAMTS1 |
| | (SEQ ID NO: 47) | | 33714308-33714327 | | | 2 (Intron) |
| OT9 | TTCAGGATATTCTATGGGA | 1, Del 12 | Chr8: | + | 1.56 | RNF139- |
| | (SEQ ID NO: 48) | | 125477293-125477311 | | | AS1 |
| | | | | | | (IncRNA, |
| | | | | | | exon) |
| OT10 | TTCAGGATCATCTATGGGA | 1, Del 10 | Chr13: | + | 1.56 | Intergenic |
| | (SEQ ID NO: 49) | | 65533459-65533477 | | | |

### Efficient Reversion of the IVS2-1 (G>A) Mutation in β-Thalassemic HSPCs Corrects Ineffective Erythropoiesis

In β-thalassemia, α- and β-globin chain imbalance causes premature death via apoptosis of erythroid precursors, thus leading to ineffective erythropoiesis, a hallmark of the disease³⁶. The typical delayed erythroid differentiation of β-thalassemic cells was corrected by our treatment, as evaluated by the flow cytometry analysis of different erythroid markers throughout the differentiation. Indeed, the early erythroid markers CD36 and CD71, were properly downregulated at the end of the differentiation in samples derived from edited HSPCs, similarly to healthy donor samples (FIG. 5A-C). Moreover, in all the samples, we observed an increased enucleation rate (frequency of DRAQ5⁻ cells) along the differentiation compared to controls. At the end of the differentiation, treated samples reached enucleation levels similar to the frequencies observed in healthy donor erythroid populations (FIG. 5D). Furthermore, the size of enucleated cells (typically reduced in β-thalassemic cells in culture) was increased in edited samples (FIG. 5E). Finally, we evaluated the potential of our treatment to rescue the apoptosis in β-thalassemic cells. Measurement of Annexin⁺ cells by flow cytometry showed a reduced apoptotic rate in edited β-thalassemic samples, as compared to the control cells (FIG. 5F).

In conclusion, we demonstrated that reverting the IVS2-1 (G>A) mutation using base editing corrected in vitro the β-thalassemic cell phenotype in terms of erythroid differentiation, enucleation, RBC size and apoptosis.

### REFERENCES

Throughout this application, various references describe the state of the art to which this invention pertains. The disclosures of these references are hereby incorporated by reference into the present disclosure.
1. Modell B, Darlison M. Global epidemiology of haemoglobin disorders and derived service indicators. Bull. World Health Organ. 2008; 86(6):480-487.
2. Taher A T, Weatherall D J, Cappellini M D. Thalassaemia. Lancet Lond. Engl. 2018; 391(10116):155-167.
3. Ithanet.eu. IthaGenes, IthaID: 200. IthaGenes IthaID 200. 2022;
4. Treisman R, Proudfoot N J, Shander M, Maniatis T. A single-base change at a splice site in a β0-thalassemic gene causes abnormal RNA splicing. Cell. 1982; 29(3):903-911.
5. Cavazzana M, Antoniani C, Miccio A. Gene Therapy for β-Hemoglobinopathies. Mol. Ther. 2017; 25(5):1142-1154.
6. Chin J Y, Kuan J Y, Lonkar P S, et al. Correction of a splice-site mutation in the beta-globin gene stimulated by triplex-forming peptide nucleic acids. Proc. Natl. Acad. Sci. 2008; 105(36):13514-13519.
7. Park S H, Lee C M, Dever D P, et al. Highly efficient editing of the β-globin gene in patient-derived hematopoietic stem and progenitor cells to treat sickle cell disease. Nucleic Acids Res. 2019; 47(15):7955-7972.
8. Milyavsky M, Gan O I, Trottier M, et al. A Distinctive DNA Damage Response in Human Hematopoietic Stem Cells Reveals an Apoptosis-Independent Role for p53 in Self-Renewal. Cell Stem Cell. 2010; 7(2):186-197.
9 Cromer M K, Vaidyanathan S, Ryan D E, et al. Global Transcriptional Response to CRISPR/Cas9-AAV6-Based Genome Editing in CD34+ Hematopoietic Stem and Progenitor Cells. Mol. Ther. J. Am. Soc. Gene Ther. 2018; 26(10):2431-2442.
10. Schiroli G, Conti A, Ferrari S, et al. Precise Gene Editing Preserves Hematopoietic Stem Cell Function following Transient p53-Mediated DNA Damage Response. Cell Stem Cell. 2019; 24(4):551-565.e8.
11. Haapaniemi E, Botla S, Persson J, Schmierer B, Taipale J. CRISPR-Cas9 genome editing induces a p53-mediated DNA damage response. Nat. Med. 2018; 24(7):927-930.
12. Kosicki M, Tomberg K, Bradley A. Repair of double-strand breaks induced by CRISPR-Cas9 leads to large deletions and complex rearrangements. Nat. Biotechnol. 2018; 36(8):765-771.
13. Boutin J, Rosier J, Cappellen D, et al. CRISPR-Cas9 globin editing can induce megabase-scale copy-neutral losses of heterozygosity in hematopoietic cells. Nat. Commun. 2021; 12(1):4922.
14. Leibowitz M L, Papathanasiou S, Doerfler P A, et al. Chromothripsis as an on-target consequence of CRISPR-Cas9 genome editing. Nat. Genet. 2021; 53(6):895-905.
15. Turchiano G, Andrieux G, Klermund J, et al. Quantitative evaluation of chromosomal rearrangements in gene-edited human stem cells by CAST-Seq. Cell Stem Cell. 2021; 28(6):1136-1147.e5.
16. Zeng J, Wu Y, Ren C, et al. Therapeutic base editing of human hematopoietic stem cells. Nat. Med. 2020; 26(4):535-541.
17. Rees H A, Liu D R. Base editing: precision chemistry on the genome and transcriptome of living cells. Nat. Rev. Genet. 2018; 19(12):770-788.
18. Antoniou P, Miccio A, Brusson M. Base and Prime Editing Technologies for Blood Disorders. Front. Genome Ed. 2021; 3:618406.
19. Richter M F, Zhao K T, Eton E, et al. Phage-assisted evolution of an adenine base editor with improved Cas domain compatibility and activity. Nat. Biotechnol. 2020; 38(7):883-891.
20. Walton R T, Christie K A, Whittaker M N, Kleinstiver B P. Unconstrained genome targeting with near-PAMless CRISPR-Cas9 engineered variants. Science. 2020; 368(6488):290-296.
21. Giarratana M-C, Kobari L, Lapillonne H, et al. Ex vivo generation of fully mature human red blood cells from hematopoietic stem cells. Nat. Biotechnol. 2005; 23(1):69-74.
22. Weber L, Frati G, Felix T, et al. Editing a γ-globin repressor binding site restores fetal hemoglobin synthesis and corrects the sickle cell disease phenotype. Sci. Adv. 2020; 6(7):.
23. Xu S, Luk K, Yao Q, et al. Editing aberrant splice sites efficiently restores β-globin expression in β-thalassemia. Blood. 2019; 133(21):2255-2262.
24. Kluesner M G, Nedveck D A, Lahr W S, et al. EditR: A Method to Quantify Base Editing from Sanger Sequencing. CRISPR J. 2018; 1:239-250.
25. Brinkman E K, Chen T, Amendola M, van Steensel B. Easy quantitative assessment of genome editing by sequence trace decomposition. Nucleic Acids Res. 2014; 42(22):e168.
26. Cradick T J, Qiu P, Lee C M, Fine E J, Bao G. COSMID: A Web-based Tool for Identifying and Validating CRISPR/Cas Off-target Sites. Mol. Ther. Nucleic Acids. 2014; 3(12):e214.
27. Clement K, Rees H, Canver M C, et al. CRISPResso2 provides accurate and rapid genome editing sequence analysis. Nat. Biotechnol. 2019; 37(3):224-226.
28. Alateeq S, Ovchinnikov D, Tracey T, et al. Identification of on-target mutagenesis during correction of a beta-thalassemia splice mutation in iPS cells with optimised CRISPR/Cas9-double nickase reveals potential safety concerns. APL Bioeng. 2018; 2(4):046103.
29. Ren Q, Sretenovic S, Liu S, et al. PAM-less plant genome editing using a CRISPR-SpRY toolbox. Nat. Plants. 2021; 7(1):25-33.
30. rs10768683 RefSNP Report—dbSNP—NCBI.
31. Renoux C, Feray C, Joly P, Lacan P, Francina A. Description of Three New α Variants and Four New β Variants: Hb Montluel [α110(G17)Ala→Val; HBA1: c.332C>T], Hb Cap d'Agde [α131(H14)Ser→Cys; HBA2: c.395C>G] and Hb Corsica [α100(G7)Leu→Pro; HBA1: 302T>C]; Hb Nîmes [β104(G6)Arg→Gly; HBB: c.313A>G], Hb Saint Marcellin [β112(G14)Cys→Gly; HBB: c.337T>G], Hb Saint Chamond [β80(EF4)Asn→0; HBB: c.241_243delAAC] and Hb Dompierre [β29(B11)Gly→Arg; HBB: c.88G>C]. Hemoglobin. 2015; 39(3):147-151.
32. rs1468286413 RefSNP Report—dbSNP—NCBI.
33. Kulcsár PI, Tálas A, Ligeti Z, Krausz S L, Welker E. SuperFi-Cas9 exhibits remarkable fidelity but severely reduced activity yet works effectively with ABE8e. Nat. Commun. 2022; 13(1):6858.
34. Li J, Yu W, Huang S, et al. Structure-guided engineering of adenine base editor with minimized RNA off-targeting activity. Nat. Commun. 2021; 12(1):2287.
35. Zhang Z, Tao W, Huang S, et al. Engineering an adenine base editor in human embryonic stem cells with minimal DNA and RNA off-target activities. Mol. Ther. —Nucleic Acids. 2022; 29:502-510.
36. Musallam K M, Rivella S, Vichinsky E, Rachmilewitz E A. Non-transfusion-dependent thalassemias. Haematologica. 2013; 98(6):833-844.

1. A method of restoring production of β-globin in a eukaryotic cell carrying an IVS2-1 (G>A) mutation comprising the step of contacting the eukaryotic cell with a gene editing platform that comprises (a) at least one adenine base-editor (ABE) and (b) at least one guide RNA (gRNA) molecule for guiding the adenine base-editor to at least one target sequence comprising the IVS2-1 (G>A) mutation and thereby restoring the production of β-globin in the eukaryotic cell.

2. The method of claim 1 wherein the eukaryotic cell is selected from the group consisting of a hematopoietic progenitor cell, a hematopoietic stem cell (HSC), pluripotent cell.

3. The method of claim 1 wherein the eukaryotic cell is homozygous or heterozygous for the IVS2-1 (G>A) mutation.

4. The method of claim 1 wherein the at least one adenine base-editor comprises a defective CRISPR/Cas nuclease.

5. The method of claim 4 wherein the defective CRISPR/Cas nuclease is a nickase.

6. The method of claim 5 wherein the nickase comprises the amino acid sequence as set forth in SEQ ID NO: 2 or SEQ ID NO:3.

7. The method of claim 1 wherein the at least one adenine base-editor comprises a non-nuclease DNA modifying enzyme that is an adenosine deaminase.

8. The method of claim 1 wherein the at least one adenine base-editor consists of the amino acid sequence as set forth in SEQ ID NO:8 (SpRY-ABE8e) or SEQ ID NO:9 (NG-ABE8e).

9. The method of claim 1 wherein the at least one guide RNA molecule targets a sequence selected from Table 1.

10. The method of claim 1 wherein the gene editing platform comprises a) the adenine base-editor NG-ABE8e or SpRY-ABE8e and b) and at least one gRNA molecule that targets a sequence selected from Table 1.

11. The method of claim 1 wherein components of the gene editing platform of the present invention are provided to the eukaryotic cell via ribonucleoprotein (RNP) complexes.

12. The method of claim 1 wherein components of the gene editing platform are provided to the eukaryotic cell via an RNA-encoded system.

13. A method of treating β-thalassemia in a subject in need thereof, the method comprising transplanting a therapeutically effective amount of a population of eukaryotic cells obtained by the method of claim 1.

14. The method of claim 13 wherein the population of eukaryotic cells is autologous to the subject.

15. The method of claim 13 wherein the patient suffers from sickle β-thalassemia.

16. The method of claim 2 wherein the pluripotent cell is an embryonic stem cell (ES) or an induced pluripotent stem cell (iPS).

17. The method of claim 5 wherein the nickase is Cas9 nickase.

18. The method of claim 17, wherein the Cas9 nickase is from S. pyogenes and has mutation D10A or H840A.


SEQUENCE LISTING (66 sequences)

SEQ ID NO: 1 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1368 aa, synthetic construct; identical to SEQ ID NO: 1

SEQ ID NO: 3 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 167 aa, Staphylococcus aureus
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 5 - protein, 5 aa, synthetic construct
GGGGS

SEQ ID NO: 6 - protein, 5 aa, synthetic construct
EAAAK

SEQ ID NO: 7 - protein, 16 aa, synthetic construct
SGSETPGTSESATPES

SEQ ID NO: 8 - protein, 1877 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGSPKKKRKV

SEQ ID NO: 9 - protein, 1624 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEVYGDYKVYDVRKMIAKSEQEEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSKRTADGSEFEPKKKRKSGGSKRTADGSEFEPKKKRK

SEQ ID NO: 10 - DNA, 20 bp, synthetic construct
  source 1..20: mol_type = other DNA
tcaggatgagtctatgggac

SEQ ID NO: 11 - DNA, 20 bp, synthetic construct
  source 1..20: mol_type = other DNA
ttcaggatgagtctatggga

SEQ ID NO: 12 - DNA, 20 bp, synthetic construct
  source 1..20: mol_type = other DNA
cttcaggatgagtctatggg

SEQ ID NO: 13 - DNA, 37 bp, Homo sapiens
  source 1..37: mol_type = genomic DNA
gagaacttcaggatgagtctatgggacgcttgatgtt

SEQ ID NO: 14 - DNA, 51 bp, synthetic construct
  source 1..51: mol_type = other DNA
gcagcgtcagatgtgtataagagacaggtaccctgttacttatccccttcc

SEQ ID NO: 15 - DNA, 48 bp, synthetic construct
  source 1..48: mol_type = other DNA
tgggctcggagatgtgtataagagacagctcaagggcacctttgccac

SEQ ID NO: 16 - DNA, 50 bp, synthetic construct
  source 1..50: mol_type = other DNA
gcagcgtcagatgtgtataagagacagagtcctagtcagagaaattgggc

SEQ ID NO: 17 - DNA, 53 bp, synthetic construct
  source 1..53: mol_type = other DNA
tgggctcggagatgtgtataagagacagagtcattttaatccagtccagaagt

SEQ ID NO: 18 - DNA, 51 bp, synthetic construct
  source 1..51: mol_type = other DNA
gcagcgtcagatgtgtataagagacagctgtgttgaatgctttctggtggc

SEQ ID NO: 19 - DNA, 50 bp, synthetic construct
  source 1..50: mol_type = other DNA
tgggctcggagatgtgtataagagacaggaccaattggtcagaggccaag

SEQ ID NO: 20 - DNA, 51 bp, synthetic construct
  source 1..51: mol_type = other DNA
gcagcgtcagatgtgtataagagacagtacagggaagtccgtttcactcag

SEQ ID NO: 21 - DNA, 52 bp, synthetic construct
  source 1..52: mol_type = other DNA
tgggctcggagatgtgtataagagacagacgtggctgtgtacaaaagtctgc

SEQ ID NO: 22 - DNA, 57 bp, synthetic construct
  source 1..57: mol_type = other DNA
gcagcgtcagatgtgtataagagacagaactctccaaaaaggcagcagtaaaacagc

SEQ ID NO: 23 - DNA, 51 bp, synthetic construct
  source 1..51: mol_type = other DNA
tgggctcggagatgtgtataagagacagttccctgtaacttggcaaggagc

SEQ ID NO: 24 - DNA, 50 bp, synthetic construct
  source 1..50: mol_type = other DNA
gcagcgtcagatgtgtataagagacagatgcctcagtacagtacccctgg

SEQ ID NO: 25 - DNA, 51 bp, synthetic construct
  source 1..51: mol_type = other DNA
tgggctcggagatgtgtataagagacaggctgtggaggaaaaagacactgc

SEQ ID NO: 26 - DNA, 47 bp, synthetic construct
  source 1..47: mol_type = other DNA
gcagcgtcagatgtgtataagagacagatgtgtcattttggctgggc

SEQ ID NO: 27 - DNA, 49 bp, synthetic construct
  source 1..49: mol_type = other DNA
tgggctcggagatgtgtataagagacaggccttttgttgtagtcagggc

SEQ ID NO: 28 - DNA, 49 bp, synthetic construct
  source 1..49: mol_type = other DNA
gcagcgtcagatgtgtataagagacagtgatgaggccaaggacctggtt

SEQ ID NO: 29 - DNA, 52 bp, synthetic construct
  source 1..52: mol_type = other DNA
tgggctcggagatgtgtataagagacaggagctgtgaaagcaaccgtacact

SEQ ID NO: 30 - DNA, 50 bp, synthetic construct
  source 1..50: mol_type = other DNA
gcagcgtcagatgtgtataagagacagcctacagctgcctatatgttaca

SEQ ID NO: 31 - DNA, 49 bp, synthetic construct
  source 1..49: mol_type = other DNA
tgggctcggagatgtgtataagagacagtccttttcttccgcatccttg

SEQ ID NO: 32 - DNA, 50 bp, synthetic construct
  source 1..50: mol_type = other DNA
gcagcgtcagatgtgtataagagacagccttgtgattctcccattggagc

SEQ ID NO: 33 - DNA, 52 bp, synthetic construct
  source 1..52: mol_type = other DNA
tgggctcggagatgtgtataagagacagccagttttcagtgggatccatagc

SEQ ID NO: 34 - DNA, 51 bp, synthetic construct
  source 1..51: mol_type = other DNA
gcagcgtcagatgtgtataagagacaggttgaactccaaagtgcagaaggc

SEQ ID NO: 35 - DNA, 52 bp, synthetic construct
  source 1..52: mol_type = other DNA
tgggctcggagatgtgtataagagacagcctggtgcctgtaattataccagc

SEQ ID NO: 36 - DNA, 19 bp, synthetic construct
  source 1..19: mol_type = other DNA
cctgtcctctgcctctgcc

SEQ ID NO: 37 - DNA, 19 bp, synthetic construct
  source 1..19: mol_type = other DNA
ggattgccaaaacggtcac

SEQ ID NO: 38 - DNA, 21 bp, synthetic construct
  source 1..21: mol_type = other DNA
cggtcaacttcaagctcctaa

SEQ ID NO: 39 - DNA, 20 bp, synthetic construct
  source 1..20: mol_type = other DNA
acagaagccaggaacttgtc

SEQ ID NO: 40 - DNA, 20 bp, synthetic construct
  source 1..20: mol_type = other DNA
ttctggaggagtctatggga

SEQ ID NO: 41 - DNA, 20 bp, synthetic construct
  source 1..20: mol_type = other DNA
ttctggatgagcctatggga

SEQ ID NO: 42 - DNA, 19 bp, synthetic construct
  source 1..19: mol_type = other DNA
ttcagcagagtctatggga

SEQ ID NO: 43 - DNA, 20 bp, synthetic construct
  source 1..20: mol_type = other DNA
ttcaggatgagtttatggga

SEQ ID NO: 44 - DNA, 21 bp, synthetic construct
  source 1..21: mol_type = other DNA
tgcaggagtgagtctatggga

SEQ ID NO: 45 - DNA, 20 bp, synthetic construct
  source 1..20: mol_type = other DNA
ttcaggatgtttctatggga

SEQ ID NO: 46 - DNA, 21 bp, synthetic construct
  source 1..21: mol_type = other DNA
ttaaggatgcagtctatggga

SEQ ID NO: 47 - DNA, 20 bp, synthetic construct
  source 1..20: mol_type = other DNA
tttaggatgagtcgatggga

SEQ ID NO: 48 - DNA, 19 bp, synthetic construct
  source 1..19: mol_type = other DNA
ttcaggatattctatggga

SEQ ID NO: 49 - DNA, 19 bp, synthetic construct
  source 1..19: mol_type = other DNA
ttcaggatcatctatggga

SEQ ID NO: 50 - DNA, 40 bp, synthetic construct
  source 1..40: mol_type = other DNA
cctgagaacttcaggatgagtctatgggacgcttgatgtt

SEQ ID NO: 51 - DNA, 40 bp, synthetic construct
  source 1..40: mol_type = other DNA
tcctgagaacttcaggatgagtctatgggacgcttgatgt

SEQ ID NO: 52 - DNA, 40 bp, synthetic construct
  source 1..40: mol_type = other DNA
tcctgagaacttcggggtgagtctatgggacccttgatgt

SEQ ID NO: 53 - DNA, 40 bp, synthetic construct
  source 1..40: mol_type = other DNA
tcctgagaacttcagggtgagtctatgggacccttgatgt

SEQ ID NO: 54 - DNA, 40 bp, synthetic construct
  source 1..40: mol_type = other DNA
tcctgagaacttcaggatgagtctatgggacccttgatgt

SEQ ID NO: 55 - DNA, 40 bp, synthetic construct
  source 1..40: mol_type = other DNA
tcctgagaacttcggggtgggtctatgggacccttgatgt

SEQ ID NO: 56 - DNA, 18 bp, synthetic construct
  source 1..18: mol_type = other DNA
tcctgagaacttcgatgt

SEQ ID NO: 57 - DNA, 40 bp, synthetic construct
  source 1..40: mol_type = other DNA
tcctgagaacttcagggtgggtctatgggacccttgatgt

SEQ ID NO: 58 - DNA, 40 bp, synthetic construct
  source 1..40: mol_type = other DNA
tcctgagaacttcgggatgagtctatgggacccttgatgt

SEQ ID NO: 59 - DNA, 40 bp, synthetic construct
  source 1..40: mol_type = other DNA
acgtggatcctgagaacttcaggatgagtctatgggacgc

SEQ ID NO: 60 - DNA, 40 bp, synthetic construct
  source 1..40: mol_type = other DNA
acgtggatcctgagaacttcggggtgagtctatgggaccc

SEQ ID NO: 61 - DNA, 40 bp, synthetic construct
  source 1..40: mol_type = other DNA
acgtggatcctgagaacttcagggtgagtctatgggaccc

SEQ ID NO: 62 - DNA, 40 bp, synthetic construct
  source 1..40: mol_type = other DNA
acgtggatcctgagaacttcaggatgagtctatgggaccc

SEQ ID NO: 63 - DNA, 40 bp, synthetic construct
  source 1..40: mol_type = other DNA
acgtggatcctgagaacttcggggtgggtctatgggaccc

SEQ ID NO: 64 - DNA, 40 bp, synthetic construct
  source 1..40: mol_type = other DNA
acgtggatcctgagaacttcagggtgggtctatgggaccc

SEQ ID NO: 65 - DNA, 40 bp, synthetic construct
  source 1..40: mol_type = other DNA
acgtggatcctgagaacttcgggatgagtctatgggaccc

SEQ ID NO: 66 - DNA, 36 bp, synthetic construct
  source 1..36: mol_type = other DNA
acgtggatcctgagaacttcaggatctatgggaccc